(19) United States
(12) Patent Application Publication
(10) Pub. No.: US 2023/0332151 A1
(43) Pub. Date: Oct. 19, 2023

(54) TREATMENT AND DETECTION OF LIVER FIBROSIS AND LIVER DISEASE USING MICRORNA

(71) Applicant: The Trustees of Columbia University in the City of New York, New York, NY (US)

(72) Inventors: Fatemeh Momen HERAVI, Teaneck, NJ (US); Akiva MINTZ, Paramus, NJ (US)

(21) Appl. No.: 18/298,287

(22) Filed: Apr. 10, 2023

Related U.S. Application Data
(63) Continuation of application No. PCT/US2021/054102, filed on Oct. 8, 2021.
(60) Provisional application No. 63/089,148, filed on Oct. 8, 2020.

(51) Int. Cl. C12N 15/113; A61K 47/69; A61K 47/64; A61P 1/16

(52) U.S. Cl. CPC C12N 15/113; A61K 47/6425; A61K 47/6911; A61P 1/16; C12N 2310/113; C12N 2310/3231; C12N 2320/32

(57) ABSTRACT

The present disclosure relates to methods and compositions for preventing and/or treating liver fibrosis, liver cancer, and/or liver disease. In particular, the present disclosure relates methods and compositions which to decrease or inhibit microRNA found to be upregulated in liver fibrosis, liver cancer, and/or liver disease, or increase or activate microRNA found to be downregulated in liver fibrosis, liver cancer and/or liver disease. The present disclosure also relates to the use of engineered exosomes or extracellular vesicles in the methods and compositions for targeted delivery of the agents to the liver.

### CROSS REFERENCE TO RELATED APPLICATIONS

The present application is a continuation of PCT Application Serial No. PCT/US2021/054102 filed Oct. 8, 2021, which claims priority in U.S. Pat. Application Serial No. 63/089,148 filed Oct. 8, 2020, which is hereby incorporated by reference in its entirety.

### FIELD

The present disclosure relates to methods and compositions for preventing and/or treating liver fibrosis, liver cancer, and/or liver disease. In particular, the present disclosure relates methods and compositions which to decrease or inhibit microRNA found to be upregulated in liver fibrosis, liver cancer, and/or liver disease, or increase or activate microRNA found to be downregulated in liver fibrosis, liver cancer, and/or liver disease. The present disclosure further relates to the use of engineered exosomes or extracellular vesicles in the methods and compositions for targeted delivery to the liver.

### BACKGROUND

Liver homeostasis is important for normal liver function, and its disbalance leads to pathogenesis. Liver fibrosis is characterized by excessive scarring. Risk factors such as chronic alcohol use impair liver function and result in pathologic states such as alcoholic liver disease (ALD). ALD is a multifactorial disease, the spectrum of which ranges from steatosis, hepatitis, fibrosis, and cirrhosis to eventual hepatocellular carcinoma (HCC) if left untreated (Seitz et al. 2018). Previous longitudinal reports indicated that approximately 50% of heavy drinkers eventually develop liver fibrosis and cirrhosis (Rehm et al. 2009). In addition to ALD, other chronic inflammatory processes activated during liver diseases of various origins such as nonalcoholic steatohepatitis (NASH), and hepatitis C (HCV) infection (Hayashi et al. 2011; Friedman 2013; Kocabayoglu and Friedman 2013), could also lead to liver fibrosis.

Liver fibrosis is an integral part in the progression of chronic liver disease, and loss of liver function, eventually leading to liver cirrhosis, liver failure, and HCC (Ramachandran and Iredale 2012). Impairment in pathways involved in induction and resolution of inflammation, tissue repair, and deposition of extracellular matrix can lead to the development of liver fibrosis (Friedman et al. 2013). Activation of quiescent hepatic stellate cells (HSCs) and inflammatory macrophages are a central event in the pathogenesis of hepatic fibrosis (Friedman 2008).

Moreover, epithelial-mesenchymal transition (EMT) in HSCs and liver parenchymal epithelial cells also contributes to fibroblast transition (Friedman et al. 2013).

MicroRNAs (miRs) are a class of small non-coding RNAs, and have emerged as new pivotal regulators of cellular processes and function (Lee et al. 1993). The role of miR-132 has been studied in the brain, and its dysregulation was found in several neurological disorders (Johnson and Buckley 2009; Wanet et al. 2012). Emerging studies suggest a role of miR-132 in other organs (Wanet et al. 2012). Recent studies indicate a role of miR-132 in pathogenesis of gastrointestinal cancers (Liu et al. 2017). miR-132 and miR-212, are derived from the same non-coding gene, share the same seed sequence, and are similarly highly conserved among vertebrates (Wanet et al. 2012), however miR-132 is unique in its regulation of numerous chromatin-remodeling factors involved in acetylation, deacetylation, and oxidative stress (Strum et al. 2009; Alvarez-Saavedra et al. 2010).

Exosomes are nanosized (50-150 nm) membrane bounded vesicles secreted by almost all types of cells in the cellular microenvironment and are found in biofluids (Momen-Heravi et al. 2013; Momen-Heravi et al. 2018; Lotvall et al. 2014). They naturally carry biomacromolecules — including different RNAs (mRNAs, regulatory miRNAs), DNAs, lipids, and proteins — and can efficiently deliver their cargoes to recipient cells, eliciting functions, and mediating cellular communications (Thery et al. 2002). Previous work by this group and by others have shown the following advantages of using exosomes for drug delivery: 1) exosomes are small and have a high efficiency for delivery due to their similarity to cell membranes; 2) exosomes are biocompatible, non-immunogenic, and non-toxic, even in repeated in vivo injections (Momen-Heravi et al. 2014); 3) exosomes are stable even after several freeze and thaw cycles, and their lipid bilayer protects the protein and RNA cargoes from enzymes such as proteases and RNases (Momen-Heravi et al. 2018); 4) exosomes have slightly negative zeta potential, leading to long circulation (Malhotra et al. 2016); and 5) exosomes also exhibit an increased capacity to escape degradation or clearance by immune system (Hood 2016).

### SUMMARY

The current disclosure provides for methods and compositions for the treatment and/or prevention of liver fibrosis, liver cancer and liver disease. The methods and compositions utilize agents which reduce liver fibrosis. In some embodiments, the methods and compositions also utilize engineered exosomes or extracellular vesicles which target delivery of the agents to the liver.

The agents for use in the methods and composition are ones which decrease or inhibit microRNA (miRNA, miR) found to be upregulated in liver fibrosis, liver cancer, and/or liver disease, including, but not limited to miR-21, miR-29, miR-33, miR-34a, miR-103/107, miR-122, miR-132, miR-181a, miR-192, miR-221/222, miR-375, and miR-802. In some embodiments, the miRNA is miR-132. Agents which decrease or inhibit microRNA include but are not limited to nucleic acids and small molecules and combinations thereof. In some embodiments, the agent is an antisense nucleic acid, a locked nucleic acid (LNA), RNA interference (RNAi), small interfering RNA (siRNA), a microRNA inhibitor, other types of modified nucleic acids, a CAS/CRISPR inhibition for miRNAs, and combinations thereof. In some embodiments, the agents which inhibit microRNA are a combination of siRNA and another agent which inhibits microRNA.

In a further embodiment, the agents are ones which increase or activate microRNA (miRNA, miR) found to be downregulated in liver fibrosis, liver cancer and/or liver disease, including but not limited to miR-146b, miR-148a, miR-181d, and miR-197. Agents which increase or activate miRNA include but are not limited to nucleic acids and small molecules and combinations thereof. In some embodiments, the agent which increases or activates microRNA is a microRNA, a microRNA mimic (modified or unmodified), a locked nucleic acid (LNA), RNA interference (RNAi), small interfering RNA (siRNA), a dCAS/CRISPR activation system for miRNAs, an agent which increases the stability of miRNAs, and combinations thereof. In some embodiments, the agents which activate microRNA are a combination of siRNA and other microRNA activators including but not limited to a microRNA or a microRNA mimic.

The current disclosure also provides for an engineered exosome or extracellular vesicle containing at least one cargo or payload for decreasing or inhibiting, or increasing or activating a microRNA implicated in liver fibrosis, liver cancer, and/or liver disease, wherein the engineered exosome or extracellular vesicle is substantially devoid of endogenous nucleic acids. Engineered exosomes or extracellular vesicles substantially devoid of endogenous nucleic acids can be made as described in co-owned application no. PCT/US2020/047894, which is incorporated herein in its entirety by reference thereto.

In some embodiments, the engineered exosome or extracellular vesicle further comprises at least one targeting moiety or a therapeutic molecule expressed on the surface of the exosome or extracellular vesicle. In some embodiments, the engineered exosome or extracellular vesicle targets liver tissue. In some embodiments, the targeting moiety comprises an integrin, a laminin, an antibody, an antibody fragment, a receptor, a component of extracellular matrix, and/or a peptide. In some embodiments, the targeting moiety is an integrin. In some embodiments, the integrin is αvβ5. In some embodiments, the targeting moiety is an asialoglycoprotein.

In some embodiments, the cargo or payload of the present engineered exosome or extracellular vesicle is an agent or agents which decrease or inhibit miRNA found to be upregulated in liver fibrosis, liver cancer, and/or liver disease, including, but not limited to miR-21, miR-29, miR-33, miR-34a, miR-103/107, miR-122, miR-132, miR-181a, miR-192, miR- 221/222, miR-375, miR-802, and combinations thereof, as described herein.

In a further embodiment, the cargo or payload of the present engineered exosome or extracellular vesicle is an agent or agents which increase or activate miRNA found to be downregulated in liver fibrosis, liver cancer, and/or liver disease, including but not limited to miR-146b, miR-148a, miR-181d, miR-197, and combinations thereof, as described herein.

One embodiment of the present disclosure is an engineered exosome or extracellular vesicle comprising a cargo or payload which is an agent or agents which decreases or inhibits a microRNA upregulated or overexpressed in liver fibrosis, liver cancer, and/or liver disease. In some embodiments, the cargo or payload includes but is not limited to a nucleic acid, a small molecule and combinations thereof. In some embodiments, the cargo or payload is an antisense nucleic acid, a locked nucleic acid (LNA), RNA interference (RNAi), small interfering RNA (siRNA), a microRNA inhibitor, other types of modified nucleic acids, a CAS/CRISPR inhibition for miRNAs, and combinations thereof. In some embodiments, the cargo or payload is an antisense nucleic acid or a locked nucleic acid (LNA). In some embodiments, the cargo or payload is a combination of siRNA and another agent which inhibits microRNA. In some embodiments, the cargo or payload decreases or inhibits miR-132. In some embodiments, the cargo or payload decreases or inhibits miR-21. In some embodiments, the cargo or payload decreases or inhibits miR-29. In some embodiments, the cargo or payload decreases or inhibits miR-33. In some embodiments, the cargo decreases or inhibits miR-34a. In some embodiments, the cargo decreases or inhibits miR-103/107. In some embodiments, the cargo decreases or inhibits miR-122. In some embodiments, the cargo or payload decreases or inhibits miR-181a.

In some embodiments, the cargo or payload decreases or inhibits miR-192. In some embodiments, the cargo or payload decreases or inhibits miR- 221/222. In some embodiments, the cargo or payload decreases or inhibits mi R-375. In some embodiments, the cargo or payload decreases or inhibits miR-802. In some embodiments, the cargo or payload decreases or inhibits the miRNA alone. In some embodiments, the cargo or payload decreases or inhibits the miRNA and another gene. In some embodiments, the cargo or payload decreases or inhibits the miRNA and another non-coding RNA.

In some embodiments, the engineered exosome or extracellular vesicle further comprises a targeting moiety which targets liver. In some embodiments, the targeting moiety is an integrin. In some embodiments, the integrin is αvβ5. In some embodiments, the targeting moiety is an asialoglycoprotein.

The present disclosure further provides for an engineered exosome or extracellular vesicle comprising an anti-miR132 LNA. In some embodiments, the engineered exosome or extracellular vesicle further comprises a targeting moiety which targets liver. In some embodiments, the targeting moiety is an integrin. In some embodiments, the integrin is αvβ5. In some embodiments, the targeting moiety is an asialoglycoprotein.

Further provided by the present disclosure is an engineered exosome or extracellular vesicle comprising a targeting moiety which targets liver and a cargo or payload which is an agent or agents which increases or activates a microRNA downregulated or underexpressed in liver fibrosis, liver cancer, and/or liver disease. In some embodiments, the cargo or payload includes but is not limited to a nucleic acid, a small molecule, and combinations thereof. In some embodiments, the cargo or payload is a microRNA, a microRNA mimic (modified or unmodified), a locked nucleic acid (LNA), RNA interference (RNAi), small interfering RNA (siRNA), a dCAS/CRISPR activation system for miRNAs, an agent which increases the stability of miRNAs, and combinations thereof. In some embodiments, the cargo or payload is a combination of siRNA and other microRNA inhibitors including but not limited to an antisense nucleic acid, a LNA or a microRNA inhibitor.

In some embodiments, the cargo or payload increases or activates miR-146b. In some embodiments, the cargo or payload increases or activates miR-148a. In some embodiments, the cargo or payload increases or activates miR-181d. In some embodiments, the cargo or pay load increases or activates miR-197.

The disclosure also provides for compositions, including pharmaceutical compositions, comprising any of the agents or engineered exosomes or extracellular vesicles or other delivery vehicles described herein. The disclosure also provides for the agents or engineered exosomes or extracellular vesicles or other delivery vehicles or compositions comprising such described herein for use in preventing and/or treating liver fibrosis, liver cancer, and/or liver disease, and methods of prevention and/or treatment of liver fibrosis, liver cancer, and/or liver disease using any of the agents, engineered exosomes or extracellular vesicles, or compositions described herein.

A further embodiment of the present disclosure is a method of detecting liver fibrosis or liver disease, comprising obtaining biological tissue and/or bodily fluid from a subject, purifying and isolating RNA from said biological tissue, and detecting the amount or level of microRNA, including but not limited miR-132 in the purified and isolated RNA sample. The amount or level of microRNA, e.g., miR-132, is compared to a reference amount or level of the microRNA. If the amount or level of the microRNA is different, either qualitatively, e.g., by visualization, or quantitatively, to the reference amount or level of the microRNA, liver fibrosis or liver disease can be detected. In some embodiments, the reference amount or level is from a healthy control.

Additionally, as shown herein, the level of miR-132 was significantly increased in the tumor tissue compared to the control and high levels of miR-132 was associated with tumors with higher tumor grades and stages in HCC patients. Thus, a further embodiment of the present disclosure is a method of detecting tumor grade and/or survival in a patient with HCC comprising obtaining tumor tissue from a subject, purifying and isolating RNA from said tumor tissue, and detecting the amount or level of microRNA, including but not limited miR-132 in the purified and isolated RNA sample. The amount or level of miRNA, e.g., miR-132, is compared to a reference amount or level of the microRNA in tumor tissue. If the amount or level of miRNA is higher in the tumor tissue than in the reference amount or level, a higher tumor grade and/or lower survival is detected.

Once the liver fibrosis or liver disease is detected using the methods disclosed herein, methods of treating the liver fibrosis or liver disease can be utilized, including but not limited to the methods disclosed herein.

The levels of miRNA can also be used to monitor treatment in a subject with liver fibrosis or liver disease.

The compositions and methods disclosed herein are useful for liver fibrosis and liver diseases including but not limited nonalcoholic steatosis hepatitis, Alcoholic Liver Disease (ALC), hepatocellular carcinoma (HCC), alcoholic hepatitis, non-alcoholic steatohepatitis (NASH), hepatitis C viral infection (HCV), non-alcoholic fatty liver disease (NAFLD), fatty liver disease, cirrhosis, and combinations thereof.

Also provided herein are kits for practicing any of the disclosed methods.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1. Induction of miR-132 in liver fibrosis. FIG. 1A shows the level of miR-132 and miR212 in liver tissues (10 mg) of control individuals and patients with alcoholic fibrosis/cirrhosis (n=8/group). The levels of miR-132 and miR-212 were quantified using TaqMan miR real-time qPCR assay and RNU48 was used as an internal control. The bars representing the controls are on the left-hand side and the bars representing the patients are on the right-hand side. FIG. 1B shows the levels of miR-132 in liver tissues (10 mg) of HCV patients with cirrhosis and respective control individuals (n=8-10/group). The levels of miR-132 was quantified using TaqMan miR real time PCR assay and RNU48 was used as an internal control. FIG. 1C shows the level of liver miR-132 in C57BU6 male mice (n=8) which received either com oil or CCU for indicated times. Total RNA from the liver was used for miR-132 analysis as described in methods and SnoRNA-202 was used as internal control. The bars representing the mice treated with com oil are on the left-hand side and the bars representing the mice treated with CCU are on the right hand side. Data represent mean ± SEM. Mann-Whitney test was employed for statistical analysis.

FIG. 2. LNA-anti-miR-132 delivery in mice. FIG. 2A is a schematic of the experimental plan. CS7BL/6 male mice (n=8) were injected either with LNA-scrambled control or LNA-anti-miR-132 (@ 15 mg/kg) or saline intraperitoneal as shown. Some mice received either corn oil or CCI4 (i.p. ; 0.6 ml/kg of body weight) for indicated times. FIG. 2B is a graph of the level of miR-132 in the various treatment groups of mice. FIG. 2C is a graph of the level of miR-212 in the various treatment groups of mice. RNA isolated from the liver was used to determine miR-132 and miR-212 expression by real-time qPCR using Taqman microRNA assay. SnoRNA-202 was used as internal control. FIG. 2D is a graph of Image J quantification of Sirius red staining of paraffin embedded liver sections in the various groups of mice. FIG. 2E is a graph of ALT levels from plasma samples from the various groups of mice. FIG. 2F is a graph of the expression levels of collagen 1α, TIMP1, and TGFβ in RNA isolated from the liver from the various groups of mice. FIG. 2G is a graph quantifying a western blot of 20ug of whole liver lysate protein used to determine a smooth muscle actin expression. Beta actin was used as a loading control. In all of the graphs in FIG. 2, the bars representing the mice treated with corn oil are on the left-hand side and the bars representing the mice treated with CCI4 are on the right-hand side. Data represent mean ± SEM. Mann-Whitney test was employed for statistical analysis. * indicates: p<0.05 compared to oil and saline treated mice. # indicates: p<0.05 compared to LNA-scrambled control treated mice after CCI4 treatment.

FIG. 3. miR-132 inhibition prevents CCl₄-induced increase in EVs in the plasma. Mice received the treatments as described in FIG. 2A. Total number of extracellular vesicles (EVs) was measured from plasma using Nanosight as described in the methods (n=5). FIG. 3A shows the size distribution of vesicles isolated from plasma after CCl₄ treatment. FIG. 3B shows the results of NTA analysis of extracellular vesicles after miR-132 inhibition. FIG. 3C is a graph of Caspase 3 activity as determined from liver cell lysate using colorimetric assay as described in the methods. Fold change was calculated using saline oil treated mice. FIG. 3D is a graph of the expression of Foxo3 and Bim quantified by real-time PCR. 18S was used to normalize Ct values. The bars representing the mice treated with corn oil are on the left-hand side and the bars representing the mice treated with CCl₄ are on the right-hand side. Data is shown as mean ± SEM. Mann-Whitney test was employed for statistical analysis. *p<0.05 compared to mice treated with oil and saline, # p<0.05 compared to ENA-scrambled control treated mice after CCI4 treatment.

FIG. 4. miR-132 inhibition attenuates CCl₄-induced increase in inflammatory and fibrogenic gene expression. Mice received the treatments as described in FIG. 2A. FIG. 4A shows the level of expression of CD68, MCP1, IE-iβ, and Cox2 in the various groups of treated mice quantified by real-time PCR. 18S was used to normalize Ct values. FIG. 4B shows the level of expression of MMP12 quantified by real-time PCR in the various groups of treated mice. FIG. 4C is quantification of a western blot using 20ug of whole liver lysate protein to determine MMP12 protein expression. Beta actin was used as a loading control. FIG. 4D shows the level of expression of MMP2 in the various groups of treated mice. FIG. 4E is quantification of a western blot using 20ug of whole liver lysate protein to determine vimentin expression. Beta actin was used as a loading control. FIG. 4F shows SIRT1 expression as quantified by quantitative real-time PCR. In all of the graphs in FIG. 4, the bars representing the mice treated with com oil are on the left-hand side and the bars representing the mice treated with CCI4 are on the right-hand side. Data represent mean ± SEM. Mann-Whitney test was employed for statistical analysis. * indicates: p<0.05 compared to oil and saline treated mice. # indicates: p<0.05 compared to LNA-scrambled control treated mice after CCI4 treatment.

FIG. 5. miR-132 increase in Kupffer cells and hepatocytes after CCl₄ treatment. Mice received the treatments as described in FIG. 2A. Total RNA was extracted from Kupffer cells and hepatocytes and expression of miR-132 (FIG. 5A, FIG. 5C) and miR-212 (FIG. 5B, FIG. 5D) was quantified using Taqman microRNA assay. SnoRNA-202 was used as internal control. FIG. 5A shows the level of miR-132 in Kupffer cells in the various groups of mice. FIG. 5B shows the level of miR-212 in Kupffer cells in the various groups of mice. FIG. 5C shows the level of miR-132 in hepatocytes in the various groups of mice. FIG. 5D shows the level of miR-212 in hepatocytes in the various groups of mice. FIG. 5E shows the level of MMP12 measured from Kupffer cells using real time qPCR in the various groups of mice. 18 s was used to normalize Ct values. Fold change was calculated using cells isolated from saline oil treated mice. FIGS. 5F-5Ishow the results of RAW macrophages transfected with either control or miRNA-132 mimic or inhibitor. For the last 24 h of transfection, cells were either treated or not with 0.1% CCl₄ and expression of microRNA, genes or proteins determined by qPCR or ELISA. FIG. 5F shows the expression of miR-132. FIG. 5G shows the expression of SIRT1. FIG. 5H shows the expression of IL-1β mRNA. FIG. 5I shows the expression of IL-1β protein. Data is shown as mean ± SEM. *p<0.05 compared to cells isolated from saline oil treated mice and, # p<0.05 compared to cells isolated from LNA-scrambled control treated mice after CCI4 treatment.

FIG. 6. miR-132 is elevated in hepatocellular carcinoma (HCC). FIG. 6A is a graph of the level of miR-132 in HCC and normal liver tissue in TCGA data (n=369 HCC; n=49 normal).

FIG. 6B shows the direct targets of mR-132 identified based on experimentally validated miR/mRNA interaction. FIG. 6C is a graph of the correlation between miR-132 and SIRT1. FIG. 6D is a graph of the correlation between miR-132 and TGFβ. FIG. 6E is a graph of the correlation between miR-132 and TMIP-1. FIG. 6F is a graph of the correlation between miR-132 and COL4A1. FIG. 6G is a graph of the correlation between miR-132 and AFP. FIG. 6H is a graph of the correlation between miR-132 and LAMB I. FIG. 6I is a graph of the levels of miR-132 in TP53 mutant HCC and TP53 non-mutant HCC (n=107 TP53 mutant and n=260 TP53 nonmutant). FIG. 6J shows the levels of miR-132 in different stages of HCC. FIG. 6K shows the levels of miR-132 in different grades of HCC. FIG. 6L shows the Kaplan-Meier survival curves of miR-132 high tumors versus miR-132 low tumors. * indicates p<0.05.

FIG. 7. Exosome mediated delivery of miR-132 mimic successfully regulates SIRT1 and inflammatory genes expression in macrophages similar to miR-132. Control or miR-132 mimic were loaded into exosomes as described in the methods. Exosomes were added to naive RAW macrophages for 12 h and afterwards exosomes were washed off and media was replaced and cultured for 24 h. Some cells were treated with 10 ng/ml LPS for 24 h. FIG. 7A shows miR-132 levels as quantified by real time qPCR and SNORNA-202 was used to normalize Ct values. FIG. 7B shows levels of SIRT1. FIG. 7C shows levels of IL-1β. FIG. 7D shows levels of IL-1β protein. FIG. 7E shows levels of MCP-1. FIG. 7F shows levels of MCP1 protein. FIG. 7G shows levels of TGFβ. Expression was evaluated by real time qPCR and EEISA. 18S was used to normalize Ct values. Data represent mean ± SEM (n=3). one-way ANOVA was employed for statistical analysis. *, **, ***, **** indicates increasingly significant p values.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The disclosure is particularly desirable for human medical applications.

The term “patient” as used in this application means a human subject. In some embodiments, the patient is suffering from liver fibrosis, liver cancer, and/or liver disease. In some embodiments, the patient suspected of having liver fibrosis, liver cancer, and/or liver disease. In some embodiments, the patient has risk factors for liver fibrosis, liver cancer, and/or liver disease.

The term “reference amount” or “reference level” as used herein can mean an amount or a quantity of a particular protein or nucleic acid in a sample from a healthy control. A “reference amount” or “reference level” may also mean an amount or a quantity of a particular protein or nucleic acid in a sample from a patient at another time point in the disease and/or treatment. A “reference amount” or “reference level” may also mean an amount or a quantity of a particular protein or nucleic acid in a sample from a patient with liver fibrosis, liver cancer, and/or liver disease.

The term “healthy control” would be a human subject who is not suffering from liver fibrosis or liver disease. Moreover, it is preferred that the healthy control be age-matched to the subject, within a reasonable range.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “in need thereof” would be a subject known or suspected of having or being at risk of liver fibrosis, liver cancer, and/or liver disease.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

The terms “microRNA” or “miRNA” will be used interchangeably and are defined as small non-coding RNAs averaging 22 nucleotides that regulate the expression of their target mRNA transcripts.

As used herein, the term “substantially free” or “substantially devoid” is used operationally, in the context of analytical testing of the exosome or extracellular vesicle. An exosome or extracellular vesicle substantially devoid or free of endogenous nucleic acids contains no greater than 10%, no greater than 8%, no greater than 5%, no greater than 2%, or no greater than 1%, of endogenous nucleic acids.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components. A polynucleotide may also be modified after polymerization, such as by conjugation with a labeling agent.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin. Sequence identity can be determined using any of a number of publicly available sequence comparison algorithms, such as BLAST, FASTA, DNA Strider, or GCG (Genetics Computer Group, Program Manual for the GCG Package, Version 7, Madison, Wisconsin).

The terms “substantially homologous” or “substantially similar” when at least about 80%, and most preferably at least about 90 or 95%, 96%, 97%, 98%, or 99% of the nucleotides match over the defined length of the DNA sequences, as determined by sequence comparison algorithms, such as BLAST, FASTA, and DNA Strider. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the engineered exosome or extracellular vesicle) and does not negatively affect the subject to which the composition(s) are administered. The pharmaceutical compositions may comprise pharmaceutically acceptable carriers, excipients, or stabilizers in the form of lyophilized formations or aqueous solutions.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Molecular Biology

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### Abbreviations

ALD- alcoholic liver disease
EMT- epithelial-mesenchymal transition
EV- extracellular vesicle
HCC- hepatocellular carcinoma
HCV- hepatitis C
HSCs- hepatic stellate cells
KCs- Kupffer cells
LNA- locked nucleic acid
miR- miRNA or microRNA
NAFLD- non-alcoholic fatty liver disease
NASH- non-alcoholic steatohepatitis

Liver fibrosis is an integral part of the progression of chronic liver disease and a risk factor for HCC. There exists to date no successful therapy for the treatment of liver fibrosis, and the most effective treatment remains removal of the causative agent, a feat which is not always possible. Because each microRNA (miR) can potentially regulate several hundred targets (Chipman and Pasquinelli 2019), pharmacological modulation of microRNAs has the potential to treat complex diseases, including liver fibrosis, that are otherwise difficult to cure using traditional small molecule approaches. Here, employing miR-based therapy, the detrimental effects of CCl₄-induced liver injury, a robust model in the study of fibrosis, were attenuated. Significantly decreased levels of liver fibrosis were found, as characterized by both phenotypic and molecular expression. Importantly, administration of LNA-anti-miR-132 produced a sustainable protective effect against induction of liver fibrosis. LNA-anti-miR-132 provided significant protection against the induction of profibrogenic markers in the CCl₄-treated mice compared to the LNA-scrambled control.

Though studies have suggested a role for miR- 132 in liver fibrosis, this is the first study that mechanistically addresses that role. Previous studies have shown that mice treated with an antagomir-132 had attenuation in cardiac fibrosis as well as reduced renal myofibroblast proliferation resulting in slowed progression of renal fibrosis (Ucar et al. 2010; Bijkerk et al.

2016). miR- 132 is known to have a broad regulatory role in the progression of hepatic steatosis, with suppression of multiple miR- 132 targets failing to wholly mimic its phenotypic effects (Hanin et al. 2017). In this study, the CCl₄-induced liver fibrosis mouse model was used to mimic the molecular and cellular features of liver fibrosis and evaluate potential roles of miR-132 in liver fibrosis and subsequent miR-based treatments.

In vivo delivery of miRs is challenged by low bioavailability and high sensitivity of RNA-interference to enzymatic degradation (Chen et al. 2014). In the present study, using LNA technology allowed the introduction of more robust, stable, and specific inhibition of miR-132 in vivo, leading to a significant reversal of fibrosis phenotype and associated molecular changes.

Knowledge of the participation of EVs in pathogenesis of liver disease has expanded greatly in recent years (Momen-Heravi et al. 2015). Production of EVs can be induced by many factors, including extracellular stimuli such as microbial attack, cellular injuries, and other stress conditions (DeToro et al. 2015). Oxidative stress has been linked to increased levels of miR-132 as well as production/release of EVs (Haviv et al. 2018; Kucharzewska et al. 2013). In this study, numbers of EVs were found after induction of liver injury in the mouse model. Administration of LNA-anti-miR-132 was capable of attenuating EV production in CCl₄-treated mice, highlighting the efficacy of miR-132 therapy in attenuating cellular injury and preventing unwanted downstream activity of released EVs. Interestingly, CCl₄-associated inhibition of caspase 3 in LNA-anti-miR-132 treated mice resulted in a decreased production of EVs, indicating a mechanistic role of CASP3 in injury-induced production of EVs. These data indicated the utility of EVs as potential biomarkers for diagnosis of liver fibrosis as well as monitoring response to therapy.

As liver fibrosis is an established risk factor for hepatocellular carcinoma and miR-132 targets many important genes which play roles in carcinogenesis (Toyoda et al. 2020), levels of miR-132 were assessed in HCC samples in TCGA database. Interestingly, the level of miR-132 was significantly increased in the tumor tissue compared to the control and high levels of miR-132 was associated with tumors with higher tumor grades and stages. HCC patients with high tumoral levels of miR-132 had significantly lower survival compared to the patients with low levels of miR-132. These data indicated possible role of miR-132 in HCC pathogenesis and highlights utility of miR-132 as HCC biomarker.

In conclusion, the data presented in this study support miR-132 as a crucial player in liver fibrosis. miR-132 was elevated in livers of patients with alcohol-induced liver fibrosis/cirrhosis as well as in CCl₄-induced liver fibrosis mouse model. Administering LNA-anti-miR-132 attenuated liver fibrosis induced by CCl₄ as well as significantly downregulated pro-fibrotic pathway. Decreased numbers of EVs, decreased caspase 3 activity, and inactivation of fibrosis-associated pathways were found after injecting LNA-anti-miR-132 in CCl₄-induced liver fibrosis mice, indicating reduced cellular injury. An association of high levels of miR-132 with HCC and poor survival outcomes in HCC patients was also found. miR-132 could be an interesting candidate with which to develop RNA-RNA interference therapies for the prevention or treatment of liver fibrosis.

### MicroRNA Inhibitors and Activators

MicroRNAs are small non-coding RNAs averaging 22 nucleotides that regulate the expression of their target mRNA transcripts. Binding of microRNAs to their targets is specified by complementary base pairing between positions 2-8 of the microRNA and the target 3′ untranslated region (3′ UTR), an mRNA component that influences translation, stability and localization.

The results set forth herein show that microRNAs are implicated in liver fibrosis, liver cancer, and liver disease.

The agents for use in the present compositions and methods include but are not limited to agents which decrease or inhibit microRNAs upregulated or overexpressed in liver fibrosis, liver cancer, and/or liver disease including, but not limited to, miR-21, miR-29, miR-33, miR-34a, miR-103/107, miR-122, miR-132, miR-181a, miR-192, miR-221/222, miR-375, andmiR-802. Inhibition of any of these microRNAs can be used in the methods for the treatment and prevention of liver fibrosis, liver cancer, and/or liver disease. In these embodiments, the miRNA is decreased or inhibited by the methods and compositions disclosed herein. Agents which decrease or inhibit microRNA include but are not limited to nucleic acids and small molecules and combinations thereof. Inhibitory agents or molecules, including but not limited to antisense oligonucleotides, a locked nucleic acid (LNA), RNA interference (RNAi), small interfering RNA (siRNA), a microRNA inhibitor, other types of modified nucleic acids, and combinations thereof, can be designed by one of skill in the art using the sequences of the miRNA listed in Table 1. These inhibitory agents and molecules can be used as cargo or payload of the engineered exosomes or extracellular vesicles.

TABLE 1

microRNAs Upregulated or Overexpressed in Liver Fibrosis, Liver Cancer, and Liver Disease

| microRNA | Gene ID Number |
|---|---|
| miRNA-21 | 406991 |
| miR-29 | 407021 |
| miR-33 | 407039 |
| miR-34a | 407040 |
| miR-103/107 | 406855; 406896; 406901 |
| miR-122 | 406906 |
| miR-132 | 406921 |
| miR-181a | 406995 |
| miR-192 | 406967 |
| miR-221/222 | 407006; 407007 |
| miR-375 | 494324 |
| miR-802 | 768219 |

The agents for use in the present compositions and methods also include agents which increase or activate miRNAs found to be downregulated or underexpressed in liver fibrosis, liver cancer, and/or liver disease, including but not limited to miR-146b, miR-148a, miR-181d, and miR-197. Activating or increasing any of these microRNAs which are downregulated in liver fibrosis, liver cancer, and/or liver disease can be used in the methods for the treatment and prevention of liver fibrosis and/or liver disease.

In these embodiments, the miRNA is increased or activated by the methods and compositions disclosed herein. In some embodiments, the agent is a nucleic acid, a small molecule, or combinations thereof. In some embodiments, the agent which increases or activates microRNA is a microRNA. In some embodiments, miRNA with sequence homology or similarity to the sequence of the miRNAs sufficient to maintain the function is the agent. In some embodiments, the agent is a microRNA mimic which is defined as containing non-natural or artificial double stranded microRNA-like RNA fragments that are constructed to contain a sequence motif on its 5′-end that is partially complementary to the target sequence in the 3′UTR. In some embodiments, the agent is a locked nucleic acid (LNA), RNA interference (RNAi), small interfering RNA (siRNA), a dCas/CRISPR activation system for miRNAs, or an agent which increases the stability of miRNAs or combinations thereof.

These molecules can be designed by one of skill in the art using the sequences of the miRNA listed in Table 2 and used as cargo or payload of the engineered exosomes or extracellular vesicles.

TABLE 2

microRNAs Upregulated or Overexpressed in Liver Fibrosis, Liver Cancer, and Liver Disease

| microRNA | Gene ID Number |
|---|---|
| miR-146b | 574447 |
| miR-148a | 406940 |
| miR-181d | 574457 |
| miR-197 | 406974 |

One agent for use in the disclosed compositions and methods is an antisense nucleic acid sequence that is complementary to the sequence of the miRNA. The antisense oligonucleotide may be DNA or RNA or comprise synthetic analogs of ribo-deoxynucleotides. An antisense oligonucleotide can be, for example, about 7, 10, 15, 20, 25, 30, 35, 40, 45, 50, 55, 60, 65, 70, 75, 80, or more nucleotides in length.

A locked nucleic acid (LN A), also known as bridged nucleic acid (BNA), include a class of bicyclic RNA analogues in which the furanose ring in the sugar-phosphate backbone is chemically locked in an RNA mimicking N-type (C3′-endo) conformation by the introduction of a 2′-0,4′-C methylene bridge (Stenvang et al. 2012). These modifications lead to increased nuclease resistance and increased binding affinity of anti-miR oligonucleotides to their target miRs (Petersen and Wnegel 2003; Stevang and Kauppinene 2008).

“RNA interference”, or “RNAi” is a form of post-transcriptional gene silencing (“PTGS”), and comprises the introduction of, e.g., double- stranded RNA into cells. The active agent in RNAi is a long double- stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. RNAi can work in human cells if the RNA strands are provided as pre-sized duplexes of about 19 nucleotide pairs, and RNAi worked particularly well with small unpaired 3′ extensions on the end of each strand (Elbashir et al. Nature 411:494-498 (2001)).

RNAi may be small interfering RNA or siRNAs, a small hairpin RNA or shRNAs, microRNA or miRNAs, or a double-stranded RNA (dsRNA).

Small interfering RNA or siRNAs are double- stranded RNA non-coding RNA molecules, typically 20-27 base pairs in length, similar to miRNA, and operating within the RNA interference (RNAi) pathway. It interferes with the expression of specific genes with complementary nucleotide sequences by degrading mRNA after transcription, preventing translation.

CRISPR activation (CRISPRa) or CRISPR inhibition (CRISPRi) may be used in the present compositions and methods.

The CRISPR (Clustered Regularly interspaced Short Palindromic Repeats) system exploits RNA-guided DNA-binding and sequence-specific cleavage of target DNA. A guide RNA (gRNA) is complementary to a target DNA sequence. The guide RNA/Cas combination confers site specificity to the nuclease. A single guide RNA (sgRNA) contains about 20 nucleotides that are complementary to a target genomic DNA sequence and a constant RNA scaffold region. The Cas (CRISPR-associated) protein binds to the guide RNA (gRNA) or sgRNA and the target DNA to which the gRNA or sgRNA binds and introduces a double strand break. Geurts et al., Science 325:433 (2009); Mashimo et al., PLoS ONE 5:e8870 (2010); Carbery et al., Genetics 186:451-459 (2010); Tesson et al., Nat. Biotech. 29:695-696 (2011). Wiedenheft et al. Nature 482:331-338 (2012); Jinek et al. Science 337:816-821 (2012); Mali et al. Science 339:823-826 (2013); Cong et al. Science 339:819-823 (2013).

In addition to a sequence that binds to a target nucleic acid, in some embodiments, the gRNA also comprises a scaffold sequence. Expression of a gRNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gRNA may be referred to as a single guide RNA (sgRNA).

Cleavage of a gene region may comprise cleaving one or two strands at the location of the target sequence by the Cas enzyme. In one embodiment, such cleavage can result in decreased transcription of a target gene. In another embodiment, the cleavage can further comprise repairing the cleaved target polynucleotide by homologous recombination with an exogenous template or donor DNA, wherein the repair results in an insertion, deletion, or substitution of one or more nucleotides of the target polynucleotide.

CRISPRa utilizes the CRISPR technique to allow for sequence-specific activation of gene expression and/or epigenetic modifications in cells (Qi et al., (2013) Repurposing CRISPR as an RNA-guided platform for sequence-specific control of gene expression, Cell 152 (5): 1173-83; Gilbert et al., (2013) CRISPR- mediated modular RNA-guided regulation of transcription in eukaryotes. Cell 154 (2):442-51). For example, a catalytically inactive Cas enzyme, e.g., dCas9, may be used to activate genes when fused to transcription activating factors. These factors include, but are not limited to, subunits of RNA Polymerase II and traditional transcription factors, such as VP16, VP64, VPR etc. (Gilbert et al., 2014, Genome-Scale CRISPR-Mediated Control of Gene Repression and Activation, Cell 159 (3): 647-61).

CRISPRi is a transcriptional interference technique that allows for sequence-specific repression of gene expression and/or epigenetic modifications in cells (Qi et al., (2013) Repurposing CRISPR as an RNA-guided platform for sequence-specific control of gene expression. Cell 152 (5): 1173-83). CRISPRi regulates gene expression primarily on the transcriptional level. CRISPRi can sterically repress transcription, e.g., by blocking transcriptional initiation or elongation. The target sequence may be the promoter and/or exonic sequences (such as the non-template strand and/or the template strand), and/or introns (Ji et al., (2014). Specific gene repression by CRISPRi system transferred through bacterial conjugation. ACS Synthetic Biology 3 (12): 929-31). CRISPRi can also repress transcription via an effector domain. Fusing a repressor domain to a catalytically inactive Cas enzyme, e.g., dead Cas9 (dCas9), may further repress transcription. For example, the Kriippel associated box (KRAB) domain can be fused to dCas9 to repress transcription of the target gene (Gilbert et al., 2013, CRISPR-mediated modular RNA-guided regulation of transcription in eukaryotes. Cell 154 (2): 442-51).

In some embodiments, a miRNA or miRNA mimic is used.

In some embodiments, the miRNA or miRNA mimic has a nucleic acid sequence that is at least 70% identical to the nucleic acid sequence of nucleic acid sequence of human miR-146b (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human miR-146b). In some embodiments, the miRNA or miRNA mimic has a nucleic acid sequence that is at least 85% identical to the nucleic acid sequence of human miR-146b (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human miR-146b). In some embodiments, the miRNA or miRNA mimic has a nucleic acid sequence that is at least 90% identical to the nucleic acid sequence of human miR-146b (e.g., a nucleic acid sequence that is 90%, 91%, 92%, 93%, 94%, 95%, 96%, 97%, 98%, 99%, or 100% identical to the nucleic acid sequence of human miR-146b). In some embodiments, the miRNA or miRNA mimic has a nucleic acid sequence that is at least 95% identical to the nucleic acid sequence of human miR-146b (e.g., a nucleic acid sequence that is 95%, 96%, 97%, 98%, 99%, or 100% identical to the nucleic acid sequence of human miR-146b).

In some embodiments, the miRNA or miRNA mimic has a nucleic acid sequence that is at least 70% identical to the nucleic acid sequence of nucleic acid sequence of human miR-148a (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human miR-148a). In some embodiments, the miRNA or miRNA mimic has a nucleic acid sequence that is at least 85% identical to the nucleic acid sequence of human miR-148a (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human miR-148a). In some embodiments, the miRNA or miRNA mimic has a nucleic acid sequence that is at least 90% identical to the nucleic acid sequence of human miR-148a (e.g., a nucleic acid sequence that is 90%, 91%, 92%, 93%, 94%, 95%, 96%, 97%, 98%, 99%, or 100% identical to the nucleic acid sequence of human miR-148a). In some embodiments, the miRNA or miRNA mimic has a nucleic acid sequence that is at least 95% identical to the nucleic acid sequence of human miR-148a (e.g., a nucleic acid sequence that is 95%, 96%, 97%, 98%, 99%, or 100% identical to the nucleic acid sequence of human miR-148a).

In some embodiments, the miRNA or miRNA mimic has a nucleic acid sequence that is at least 70% identical to the nucleic acid sequence of nucleic acid sequence of human miR-181d (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human miR-18 Id). In some embodiments, the miRNA or miRNA mimic has a nucleic acid sequence that is at least 85% identical to the nucleic acid sequence of human miR-181d (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human miR-181d). In some embodiments, the miRNA or miRNA mimic has a nucleic acid sequence that is at least 90% identical to the nucleic acid sequence of human miR-181d (e.g., a nucleic acid sequence that is 90%, 91%, 92%, 93%, 94%, 95%, 96%, 97%, 98%, 99%, or 100% identical to the nucleic acid sequence of human miR-181d). In some embodiments, the miRNA or miRNA mimic has a nucleic acid sequence that is at least 95% identical to the nucleic acid sequence of human miR-181d (e.g., a nucleic acid sequence that is 95%, 96%, 97%, 98%, 99%, or 100% identical to the nucleic acid sequence of human miR-146b).

In some embodiments, the miRNA or miRNA mimic has a nucleic acid sequence that is at least 70% identical to the nucleic acid sequence of nucleic acid sequence of human miR-197 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human miR-197). In some embodiments, the miRNA or miRNA mimic has a nucleic acid sequence that is at least 85% identical to the nucleic acid sequence of human miR-197 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human miR-197). In some embodiments, the miRNA or miRNA mimic has a nucleic acid sequence that is at least 90% identical to the nucleic acid sequence of human miR-197 (e.g., a nucleic acid sequence that is 90%, 91%, 92%, 93%, 94%, 95%, 96%, 97%, 98%, 99%, or 100% identical to the nucleic acid sequence of human miR-197). In some embodiments, the miRNA or miRNA mimic has a nucleic acid sequence that is at least 95% identical to the nucleic acid sequence of human miR-197 (e.g., a nucleic acid sequence that is 95%, 96%, 97%, 98%, 99%, or 100% identical to the nucleic acid sequence of human miR-191).

### Extracellular Vesicles and Exosomes

Extracellular vesicles are membrane enclosed vesicles released by cells. Their primary constituents are lipids, proteins and nucleic acids. They are composed of a lipid-protein bilayer encapsulating an aqueous core comprising nucleic acids and soluble proteins. Extracellular vesicles include, but are not limited to, exosomes, shedding vesicles, microvesicles, small vesicles, large vesicles, microparticles, and apoptotic bodies, based on their size, cellular origin and formation mechanism. Exosomes are formed by inward budding of late endosomes forming multivesicular bodies (MVB) which then fuse with the limiting membrane of the cell concomitantly releasing the exosomes. Shedding vesicles are formed by outward budding of the limiting cell membrane followed by fusion. When a cell undergoes apoptosis, the cell disintegrates and divides its cellular content in different membrane enclosed vesicles termed apoptotic bodies. Non-limiting examples of extracellular vesicles include circulating extracellular vesicles, beta cell extracellular vesicles, islet cell extracellular vesicles, exosomes and apoptotic bodies, and combinations thereof.

Large extracellular vesicles can range from about 5 µm to about 12 µm in diameter. Apoptotic bodies can range from about 1 pm to about 5 pm in diameter. Microvesicles can range from about 100 nm to about 1 µm in diameter. Exosomes can range from about 30 nm to about 150 nm, from about 30 nm to about 100 nm, or from about 50 nm to about 150 nm in diameter or from about 50 nm to about 200 nm.

Extracellular vesicles or exosomes may be isolated or derived from bone marrow, red blood cells, tumor cells, immune cells, epithelial cells, fibroblasts, or stem cells. Extracellular vesicles or exosomes may be isolated or derived from B cells, T cells, monocytes, or macrophages. In one embodiment, extracellular vesicles or exosomes to be taken up by a specific type of cells (e.g., monocytes/macrophages) are isolated or derived from the same type of cells (e.g., monocytes/macrophages).

Extracellular vesicles or exosomes may be isolated or derived from a body fluid. For example, the body fluids can include, but are not limited to, serum, plasma, blood, whole blood and derivatives thereof, urine, tears, saliva, sweat, cerebrospinal fluid (CSF), oral mucus, vaginal mucus, seminal plasma, semen, prostatic fluid, excreta, ascites, lymph, bile, breast milk and amniotic fluid.

Extracellular vesicles or exosomes may be isolated or derived from cultured cells.

Methods for isolating extracellular vesicles include size separation methods such as centrifugation. In one embodiment, isolating various components of extracellular vesicles may be through an isolation method including sequential centrifugation. The method may include centrifuging a sample at 800 g for a desired amount of time, collecting the pellet containing cells and cellular debris and (first) supernatant, centrifuging the (first) supernatant at 2,000 g for a desired time, collecting the pellet containing large extracellular vesicles and apoptotic bodies and (second) supernatant. The sequential centrifugation method can further include centrifuging the (second) supernatant at 10,000 g, collecting the pellet containing micro vesicles and (third) supernatant. The sequential centrifugation method can further include centrifuging the (third) supernatant at 100,000 g, collecting the pellet containing exosomes (ranging from about 30 nm to about 200 nm in diameter) and (fourth) supernatant. The sequential centrifugation method can further include washing each of the pellets including the extracellular vesicles (e.g., large extracellular vesicles and apoptotic bodies, micro vesicles, and exosomes) such as in phosphate buffered saline followed by centrifugation at the appropriate gravitational force and collecting the pellet containing the extracellular vesicles. Isolation, purity, concentration, size, size distribution, and combinations thereof of the extracellular vesicles following each centrifugation step can be confirmed using methods such as nanoparticle tracking, transmission electron microscopy, immunoblotting, and combinations thereof. Nanoparticle tracking (NTA) to analyze extracellular vesicles such as for concentration and size can be performed by dynamic light scattering using commercially available instruments such as ZETAVIEW (commercially available from ParticleMetrix, Meerbusch, Germany). Following isolation, the method can further include detecting an extracellular vesicle marker.

Methods for isolating extracellular vesicles also include using commercially available reagents such as, for example, EXOQUICK TC reagent (commercially available from System Biosciences, Palo Alto, Calif.).

Exosomes are small vesicular bodies that are secreted from cells into the cellular microenvironment and biofluids and can enter both neighboring cells and the systemic circulation. Exosomes are actively assembled from intracellular multi vesicular bodies (MVBs) by the endosomal sorting complex required for transport (ESCRT) machinery. Exosomes contain various molecular constituents of their cell of origin, including, but not limited to, proteins, RNA (such as mRNA, miRNA, etc.), lipids and DNA.

Exosome may be isolated by any suitable techniques, including ultracentrifugation, micro-filtration, size-exclusion chromatography etc. or a combination thereof. Exosome can be isolated using a combination of techniques based on both physical (e.g., size, density) and biochemical parameters (e.g., presence/absence of certain proteins involved in their biogenesis). In certain embodiments, exosomes are isolated using a kit. In one embodiment, exosomes are isolated using the Total Exosome Isolation Kit and/or the Total Exosome Isolation Reagent from Invitrogen.

Following isolation, the method can further include detecting an extracellular vesicle marker of the extracellular vesicle.

Extracellular vesicle or exosome markers include CD9, CD63, CD81, LAPM1, TSG101, and combinations thereof.

To make exosomes or extracellular vesicles biologically safe and to eliminate the unwanted side effects associated with the endogenous exosomal cargo, exosomes or extracellular vesicles can be engineered to be substantially free or devoid of endogenous nucleic acids. This can result in minimal off-target effects.

To prepare exosomes or extracellular vesicles substantially devoid of endogenous nucleic acids, one or more players in sorting or loading nucleic acids into exosomes or extracellular vesicles may be downregulated or inhibited. These players include but are not limited to heterogeneous nuclear ribonucleoprotein A2B1 (hnRNPA2B1), Dorsha, Alix, major vault protein (MVP), Exportin 1 and Exportin 5. The protein hnRNPA2B1 specifically binds exosomal RNA through the recognition of specific motifs, controlling their loading into exosomes. Villarroya-Beltri et al. (2013). Thus, by knocking down hnRNPA2B1, the key player in sorting RNAs into exosomes, exosomes can be engineered to be substantially free or devoid of endogenous nucleic acids. Dorsha functions as the initiator of microRNA biogenesis by cleaving pri-miRNA to mature forms of microRNA. Alix mediates nucleic acid loading into the exosomes. Han et al. 2004; Iavello et al. 2016.

The amount/level and/or activity of hnRNPA2B1, Dorhsa, and/or Alix, etc. may be downregulated or otherwise decreased or suppressed. The mechanism of inhibition may be at the genetic level (e.g., interference with or inhibit expression, transcription or translation, etc.) or at the protein level (e.g., binding, competition, etc.).

Exosomes or extracellular vesicles may be loaded with different cargoes by, e.g., transduction, expression in producing cells, electroporation, transfection, microinjection, etc. Cultured cells may be engineered to express various cargoes by, e.g., transduction, electroporation, transfection, microinjection, etc. Exosomes or extracellular vesicles are then produced from these cells which are loaded with the desired cargo(s). The payload may be introduced into the cell in the form of a DNA (e.g., cDNA), mRNA and protein.

The nucleic acids may be delivered to cultured cells in vitro. Nucleic acids can be delivered as part of a larger construct, such as a plasmid or viral vector, or directly, e.g., by electroporation, lipid vesicles, viral transporters, microinjection, heat shock, and biolistics. Non-limiting examples of methods to introduce nucleic acids into cells include lipofectamine transfection, calcium phosphate co-precipitation, electroporation, DEAE-dextran treatment, microinjection, lipid-mediated transfection, viral infection, chemical transformation, electroporation, lipid vesicles, viral transporters, ballistic transformation, pressure induced transformation, viral transduction, particle bombardment, and other methods known in the art.

Suitable cells include, but are not limited to, mammalian cells (e.g., human cells, mouse cells, rat cells, etc.), primary cells, stem cells, immune cells, and any other type of cells known to those skilled in the art.

### Cargo or Payload

The cargo or payload of the present engineered exosome or extracellular vesicle includes but is not limited to agents and molecules which decrease or inhibit microRNAs upregulated or overexpressed in liver fibrosis, liver cancer, and/or liver disease including, but not limited to, miR-21, miR-29, miR-33, miR-34a, miR-103/107, miR-122, miR-132, miR-181a, miR-192, miR-221/222, miR-375, and miR-802, as described herein.

The cargo or payload of the present engineered exosome or extracellular vesicle also includes agents and molecules which increase or activate miRNAs found to be downregulated or underexpressed in liver fibrosis, liver cancer, and/or liver disease, including but not limited to miR-146b, miR-148a, miR-181d, and miR-197, as described herein.

### Targeting Moieties and Therapeutic Molecules

While the engineered exosomes or extracellular vesicles are taken up efficiently by the liver and are able to efficiently deliver cargo to the liver, the engineered exosome or extracellular vesicles can further comprise a targeting moiety.

The targeting moiety may be a cell-specific, tissue-specific, or organ-specific targeting moiety. The targeting moiety may be an integrin, a cell-specific, tissue-specific, or organ-specific antibody, a cell-specific, tissue-specific, or organ-specific receptor, or a cell-specific, tissue-specific, or organ-specific polypeptide/peptide. The engineered exosome or extracellular vesicle will be targeted to and taken up selectively by the desired cells/tissue/organ. In some embodiments, the targeting moiety targets a cell, tissue or organ including but not limited to the liver.

The targeting moiety may be an integrin, such as the αvβ5 integrin, the α6β4 integrin, and the α6β1 integrin, etc. In some embodiments, the exosomes express the αvβ5 integrin which specifically binds to Kupffer cells, thus delivering the engineered exosome or extracellular vesicle specifically to the liver.

In some embodiments, the targeting moiety is asialoglycoprotein.

### Exosome or Extracellular Vesicle Loading

Exosomes or extracellular vesicles may be loaded with different cargoes by, e.g., transduction, expression in producing cells, electroporation, transfection, microinjection, etc. Cultured cells may be engineered to express various cargoes by, e.g., transduction, electroporation, transfection, microinjection, etc. Exosomes or extracellular vesicles are then produced from these cells which are loaded with the desired cargo(s). The payload may be introduced into the cell in the form of a DNA (e.g., cDNA), mRNA and protein.

Proteins, peptides, or polypeptides may be loaded into an exosome or extracellular vesicle by transfection or electroporation. Proteins or polypeptides may also be introduced into a cell from which an engineered exosome or extracellular vesicle may be generated.

The nucleic acids may be delivered to cultured cells in vitro. Nucleic acids can be delivered as part of a larger construct, such as a plasmid or viral vector, or directly, e.g., by electroporation, lipid vesicles, viral transporters, microinjection, heat shock, and biolistics. Non-limiting examples of methods to introduce nucleic acids into cells include lipofectamine transfection, calcium phosphate co-precipitation, electroporation, DEAE-dextran treatment, microinjection, lipid-mediated transfection, viral infection, chemical transformation, electroporation, lipid vesicles, viral transporters, ballistic transformation, pressure induced transformation, viral transduction, particle bombardment, and other methods known in the art.

### Additional Delivery Vehicles

The delivery of the agents can be accomplished using many delivery vehicles including but not limited to a synthetic or natural delivery vehicle, such as exosomes, micro vesicles, apoptotic bodies, oncosomes, extracellular vesicles, microparticles, liposomes or nanoparticles. It can also be accomplished by delivering genetic material encoding the agents using plasmids and vectors.

### Liposomes and Nanoparticles

The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868; and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 pm. Sonication of MLVs results in the formation of small unilamellar vesicles (SUVs) with diameters in the range of 200 to 500 A, containing an aqueous solution in the core.

Alternatively, nanocapsule formulations may be used. Nanocapsules can generally entrap substances in a stable and reproducible way.

Nanoparticles are a colloidal carrier system that has been shown to improve the efficacy of an encapsulated drug by prolonging the serum half-life. Polyalkylcyanoacrylates (PACAs) nanoparticles are a polymer colloidal drug delivery system that is in clinical development. Biodegradable poly (hydroxyl acids), such as the copolymers of poly (lactic acid) (PLA) and poly (lactic-co-glycolide) (PLGA) are being extensively used in biomedical applications and have received FDA approval for certain clinical applications. In addition, nanoparticles have many desirable carrier features including: (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier has the ability to be freeze-dried and reconstituted in solution without aggregation; (iv) that the carrier be biodegradable; (v) that the carrier system be of small size; and (vi) that the carrier enhances the particles persistence. Nanoparticles may be synthesized using virtually any biodegradable shell known in the art. Such polymers are biocompatible and biodegradable and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charged nucleic acids. Nanoparticles may also be modified with polyethylene glycol (PEG), which also increases the half-life and stability of the particles in circulation. Alternatively, the COOH group may be converted to an N-hydroxy succinimide (NHS) ester for covalent conjugation to amine-modified compounds.

### Vectors

As used herein, a “vector” may be any of a number of nucleic acids into which a desired sequence or sequences may be inserted for transport between different genetic environments or for expression in a host cell. Vectors include, but are not limited to, nucleic acid molecules that are single-stranded, double- stranded, or partially double-stranded; nucleic acid molecules that comprise one or more free ends, no free ends (e.g., circular); nucleic acid molecules that comprise DNA, RNA, or both; and other varieties of polynucleotides known in the art.

Vectors include, but are not limited to, viral vectors, plasmids, cosmids, fosmids, phages, phage lambda, phagemids, and artificial chromosomes.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.”

A common type of vector is a “plasmid”, which generally is a self-contained molecule of double- stranded DNA, usually of bacterial origin, that can readily accept additional (foreign) DNA and which can readily be introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes and may be from the same or different organisms. A large number of vectors, including plasmid and fungal vectors, have been described for replication and/or expression in a variety of eukaryotic and prokaryotic hosts, and many appropriate host cells, are known to those skilled in the relevant art. Recombinant cloning vectors will often include one or more replication systems for cloning or expression, one or more markers for selection in the host, e.g. antibiotic resistance, and one or more expression cassettes.

Viral vectors may be derived from DNA viruses or RNA viruses, which have either episomal or integrated genomes after delivery to the cell. See Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149- 1154 (1988); Vigne, Restorative Neurology and Neuroscience 8:35-36 (1995); Kremer & Perricaudet, British Medical Bulletin 51(1):31-44 (1995); Haddada e/ al., in Current Topics in Microbiology and Immunology, Doerfler and Bohm (eds) (1995); and Yu et al., Gene Therapy 1:13-26 (1994).

Viral vectors may be derived from retroviruses (including lentiviruses), replication defective retroviruses (including replication defective lentiviruses), adenoviruses, replication defective adenoviruses, adeno-associated viruses (AAV), herpes simplex viruses, and poxviruses. In some embodiments, the vector is a lentiviral vector. Options for gene delivery

of viral constructs are known (see, e.g., Ausubel et al., Current Protocols in Molecular Biology, John Wiley & Sons, New York, 1989; Kay, et al., 2001 Nat. Medic. 7(1):33-40; and Walther W. and Stein U., 2000 Drugs, 60(2): 249-71).

### Pharmaceutical Compositions and Methods of Administration

The present methods include the administration of agents which decrease or inhibit microRNAs upregulated or overexpressed in liver fibrosis, liver cancer, or liver disease including but not limited to, miR-21, miR-29, miR-33, miR-34a, miR-103/107, miR-122, miR-132, miR-181a, miR-192, miR- 221/222, miR-375, and miR-802, or which increase or activate miRNAs found to be downregulated or underexpressed in liver fibrosis, liver cancer, or liver disease including but not limited to miR-146b, miR-148a, miR-181d, and miR-197.

The agent must be in the appropriate form for administration of choice.

When the agent is a nucleic acid such as DNA, RNA, interfering RNA or microRNA, methods for delivery include receptor mediated endocytosis where the nucleic acid is coupled to a targeting molecule that can bind to a specific cell surface receptor, inducing endocytosis and transfer of the nucleic acid into cells. Coupling is normally achieved by covalently linking poly-lysine to the receptor molecule and then arranging for (reversible) binding of the negatively charged nucleic acid to the positively charged poly-lysine component. Another approach utilizes the transferrin receptor or folate receptor which is expressed in many cell types. When producing the microRNA for this method of administration, the microRNA could be manufactured to have a guide strand which is identical to the microRNA of interest and a passenger strand that is modified and linked to a molecule for increasing cellular uptake.

Liposomes are spherical vesicles composed of synthetic lipid bilayers which mimic the structure of biological membranes. The nucleic acid to be transferred is packaged in vitro with the liposomes and used directly for transferring the nucleic acid to a suitable target tissue in vivo. The lipid coating allows the nucleic acid to survive in vivo, bind to cells and be endocytosed into the cells. Cationic liposomes (where the positive charge on liposomes stabilize binding of negatively charged DNA), have are one type of liposome.

The nucleic acids can also be administered with a lipid to increase cellular uptake. The nucleic acids may be administered in combination with a cationic lipid, including but not limited to, lipofectamine, DOTMA, DOPE, and DOTAP.

Other lipid or liposomal formulations including nanoparticles and methods of administration have been described as for example in U.S. Pat. Publication 2003/0203865, 2002/0150626, 2003/0032615, and 2004/0048787. Methods used for forming particles are also disclosed in U.S. Pat. Nos. 5,844,107, 5,877,302, 6,008,336, 6,077,835, 5,972,901, 6,200,801, and 5,972,900.

The present disclosure provides a pharmaceutical composition comprising the agents, or the present engineered exosomes or extracellular vesicles, or other delivery vehicles described herein. The present disclosure provides uses of the present agents or engineered exosomes or extracellular vesicles or other delivery vehicles for manufacturing a medicament for use in treating a condition or disorder.

In some embodiments, the present agents or engineered exosomes or extracellular vesicles or other delivery vehicles may be mixed with a pharmaceutically acceptable excipient or carrier to form a pharmaceutical composition.

Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington’s Pharmaceutical Sciences and U.S. Pharmacopeia: National Formulary, Mack Publishing Company, Easton, PA (1984).

Pharmaceutically acceptable carriers, including buffers, are well known in the art, and may comprise phosphate, citrate, and other organic acids, antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Therapeutically effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

The present engineered exosomes or extracellular vesicles may be delivered to a cell by contacting the cell with the exosomes or extracellular vesicles.

The present engineered exosomes or extracellular vesicles or the present composition may be delivered/administered to a subject by any route, including, without limitation, intravenous, intracerebroventricular (ICV) injection, intracisternal injection or infusion, oral, transdermal, ocular, intraperitoneal, subcutaneous, implant, sublingual, subcutaneous, intramuscular, rectal, mucosal, ophthalmic, intrathecal, intra-articular, intra-arterial, sub-arachnoid, bronchial and lymphatic administration. The present composition may be administered parenterally or systemically. The present composition may be administered locally.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer’s Injection, Dextrose Injection, Dextrose and Sodium Chloride Injection, and Lactated Ringer’s Injection; water-miscible vehicles including, but not limited to, ethyl alcohol, polyethylene glycol and polypropylene glycol; and nonaqueous vehicles including, but not limited to, corn oil, cottonseed oil, peanut oil, sesame oil, ethyl oleate, isopropyl myristate and benzyl benzoate.

Injectable forms include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils.

Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active agent in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

### Methods for Preventing And/or Treating Liver Fibrosis. Liver Cancer, and Liver Disease

The present disclosure also provides for methods of preventing and/or treating liver fibrosis, liver cancer, and liver disease comprising administering to a subject in need thereof a therapeutically effective amount of an agent or agents which decreases or inhibits microRNAs found to be upregulated in liver disease, including, but not limited to miR-21, miR-29, miR-33, miR-34a, miR-103/107, miR-122, miR-132, miR-181a, miR-192, miR-221/222, miR-375, miR-802, and combinations thereof.

The present disclosure also provides for methods of preventing and/or treating liver fibrosis, liver cancer, and liver disease comprising administering to a subject in need thereof a therapeutically effective amount of an agent or agents which increases or activates miRNA found to be downregulated in liver disease, including but not limited to miR-146b, miR-148a, miR-181d, miR-197, and combinations thereof.

The present disclosure also provides for methods of preventing and/or treating liver fibrosis, liver cancer, and liver disease comprising administering to a subject in need thereof a therapeutically effective amount of an engineered exosome or extracellular vesicle disclosed herein or a composition comprising an engineered exosome or extracellular vesicle disclosed herein.

The engineered exosome or extracellular vesicle used in the methods can comprise cargo or payloads which decrease or inhibit microRNAs found to be upregulated in liver fibrosis, liver cancer, and liver disease, including, but not limited to miR-21, miR-29, miR-33, miR-34a, miR-103/107, miR-122, miR-132, miR-181a, miR-192, miR-221/222, miR-375 miR-802, and combinations thereof.

The engineered exosome or extracellular vesicle used in the methods can comprise cargo or payloads which increase or activate miRNA found to be downregulated in liver fibrosis, liver cancer, and liver disease, including but not limited to miR-146b, miR-148a, miR-18 Id, miR-197, and combinations thereof.

The conditions to be treated by the present engineered exosome or extracellular vesicle include a variety of diseases including liver fibrosis and liver diseases including but not limited nonalcoholic steatosis hepatitis, Alcoholic Liver Disease (ALC), hepatocellular carcinoma (HCC), alcoholic hepatitis, non-alcoholic steatohepatitis (NASH), hepatitis C viral infection (HCV), non-alcoholic fatty liver disease (NAFLD), fatty liver disease, cirrhosis, and combinations thereof.

### Kits

The present disclosure also encompasses kits containing the present compositions or agents or engineered exosomes or extracellular vesicles.

In some embodiments, the kit comprises the present compositions or agent or engineered exosomes or extracellular vesicles and instructions for using the kit. Elements may be provided individually or in combinations.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form).

### Methods of Detection of Liver Fibrosis or Liver Disease or HCC Tumor Grade

As shown herein, high levels of miR-132 were found associated with both liver fibrosis and with poor outcomes of patients with HCC.

Thus, one embodiment of the present disclosure is a method of detecting liver fibrosis or liver disease in a subject, comprising:
a. detecting or measuring the amount or level of one or microRNAs in a sample from the subject; and
b. comparing the amount or level of the one or more microRNAs in the sample to a reference amount or level of one or more of the microRNAs, wherein if the amount or level of the one or more microRNAs in the sample is different than the reference amount or level of one or more microRNAs, the subject is suffering from liver fibrosis and/or liver disease, and wherein the reference amount or level is from a healthy control.

A further embodiment of the present disclosure is a method of detecting higher tumor grade and lower survival in a subject with hepatocellular carcinoma, comprising:
a. detecting or measuring the amount or level of one or microRNAs in a tumor sample from the subject; and
b. comparing the amount or level of the one or more microRNAs in the tumor sample to a reference amount or level of one or more of the microRNAs, wherein if the amount or level of the one or more microRNAs in the tumor sample is different than the reference amount or level of one or more microRNAs, the subject has a higher tumor grade and lower survival.

The levels of microRNA found in a sample can be compared to the levels of these microRNAs in healthy controls. This comparison can be done in many ways. The same assay can be performed simultaneously or consecutively, on a purified and isolated RNA sample from a healthy control and the results compared qualitatively, e.g., visually, i.e., does the RNA sample from the healthy control produce the same intensity of signal as the RNA sample from the subject in the same assay, or the results can be compared quantitatively, e.g., a value of the signal for the RNA sample from the subject is obtained and compared to a known value of the RNA in a healthy control.

A higher level of microRNA in a sample from a subject as compared to the healthy control would indicate the subject has liver fibrosis or liver disease.

Any method that detects microRNA can be used in the methods herein.

Methods begin by obtaining a sample of biological tissue or bodily fluid from the patient and extracting, isolating and/or purifying the nucleic acid (e.g., genomic DNA, cDNA, RNA) from the tissue or fluid.

The nucleic acid can be obtained from any biological tissue. Preferred biological tissues include, but are not limited to, brain, epidermal, whole blood, and plasma.

The nucleic acid can be obtained from any bodily fluid. Preferred bodily fluids include, but are not limited to, cerebrospinal fluid, plasma, saliva, sweat, and urine.

When detecting the tumor grade and/or survival outcome of a subject with HCC, the nucleic acid is obtained from tumor tissue.

The nucleic acid is extracted, isolated and purified from the cells of the tissue or fluid by methods known in the art. Methods for detecting microRNA include but are not limited to Northern blots, in situ hybridization, real time PCR, nuclease protection assays, poly-A tailed reverse transcription, microRNA amplification profiling, microRNA serial analysis of gene expression, microarrays, enzyme amplified assays, sequencing, and nanoparticle methods.

One useful technique for determining whether the microRNAs are present in a sample from a subject is small RNA sequencing and annotation using barcoded small RNA cDNA libraries and generating microRNA expression profiles of the sample.

Next generation sequencing platforms such as Genome Analyzer (Illumina Inc) or Genome Sequencer FLX (454 Life Science and Roche Applied Science) are also useful to detect microRNA by sequencing. Deep sequencing uses massively parallel sequencing, generating millions of small RNA sequence reads from a given sample.

In some embodiments, the microRNA is miR-132.

### EXAMPLES

This invention will be better understood from the Experimental Details, which follow. However, one skilled in the art will readily appreciate that the specific methods and results discussed are merely illustrative of the invention as described more fully in the claims that follow thereafter.

### Example 1 - Materials and Methods for Examples 2-7

### Animal Studies

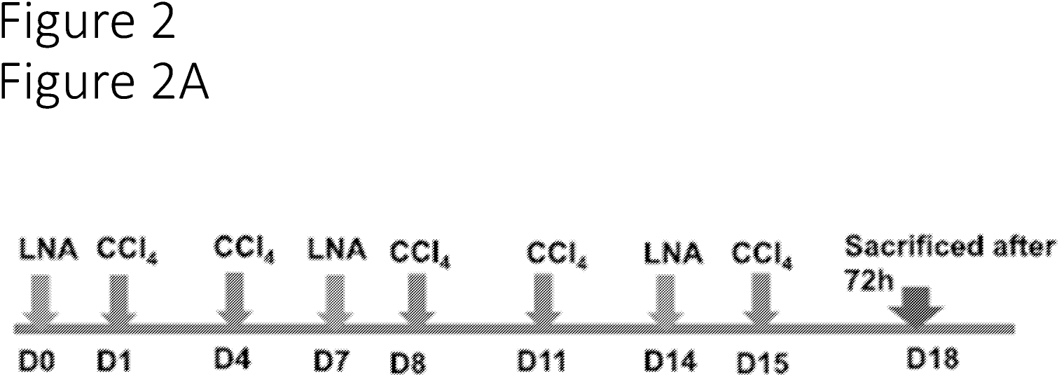

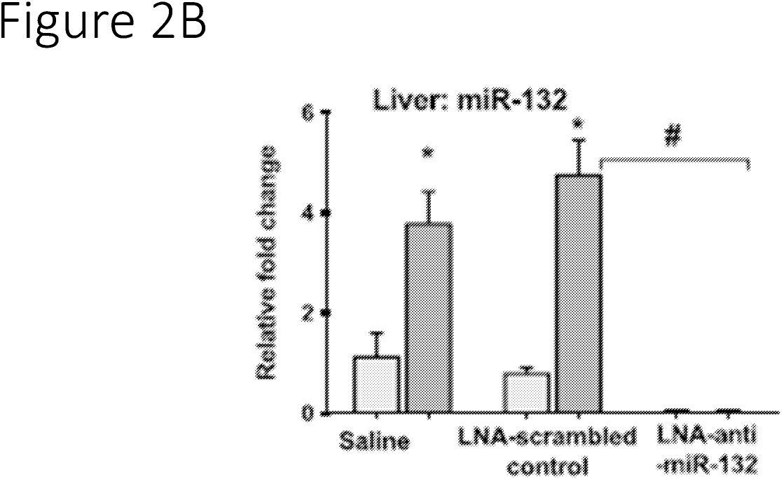

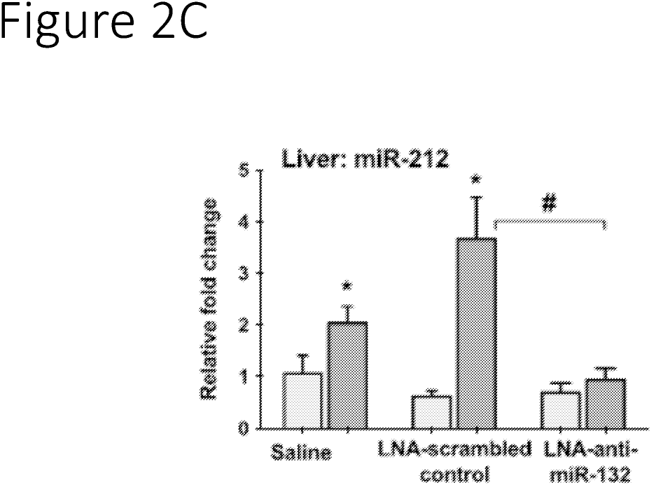

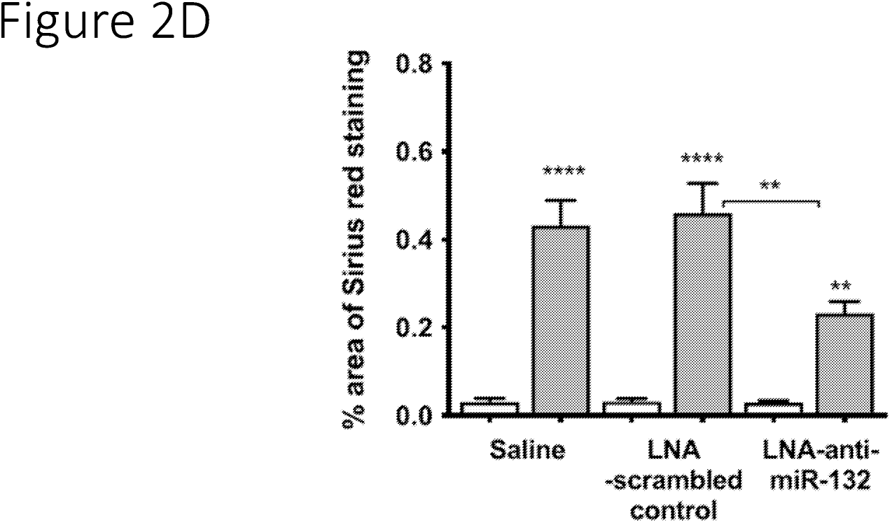

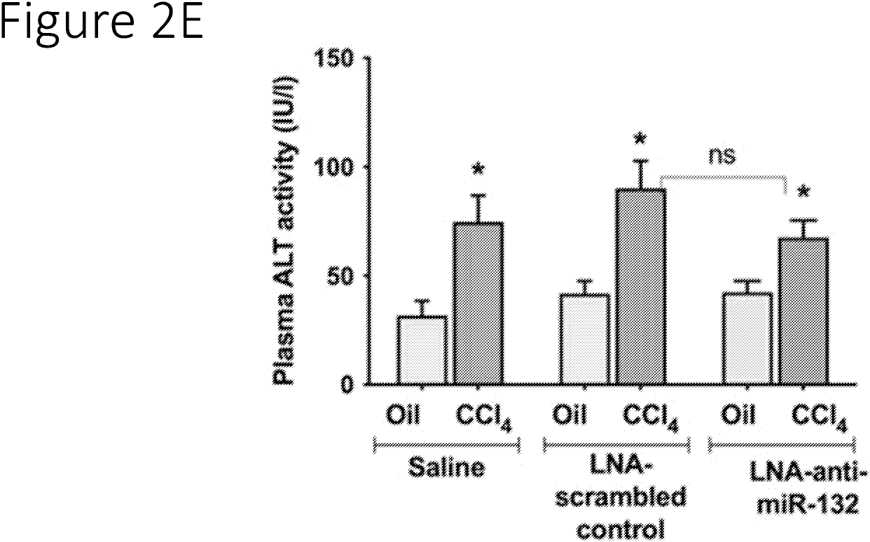

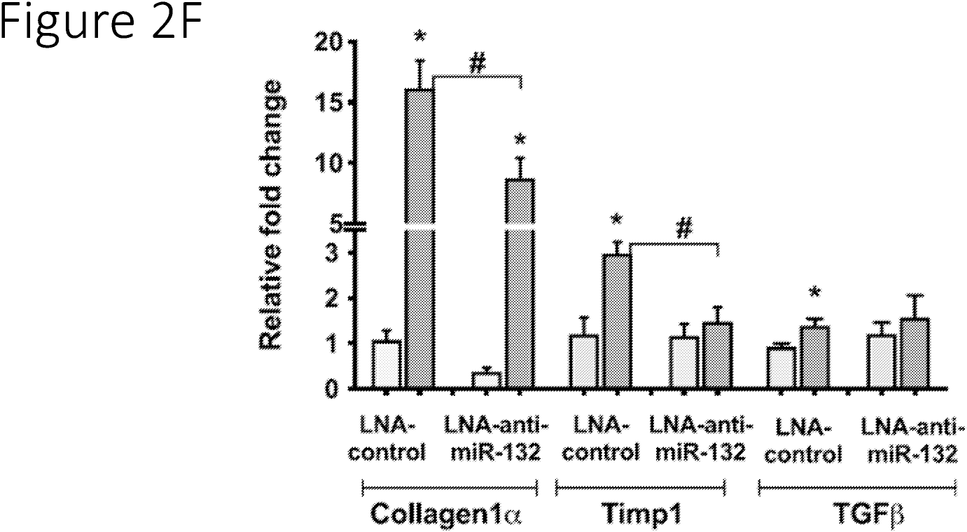

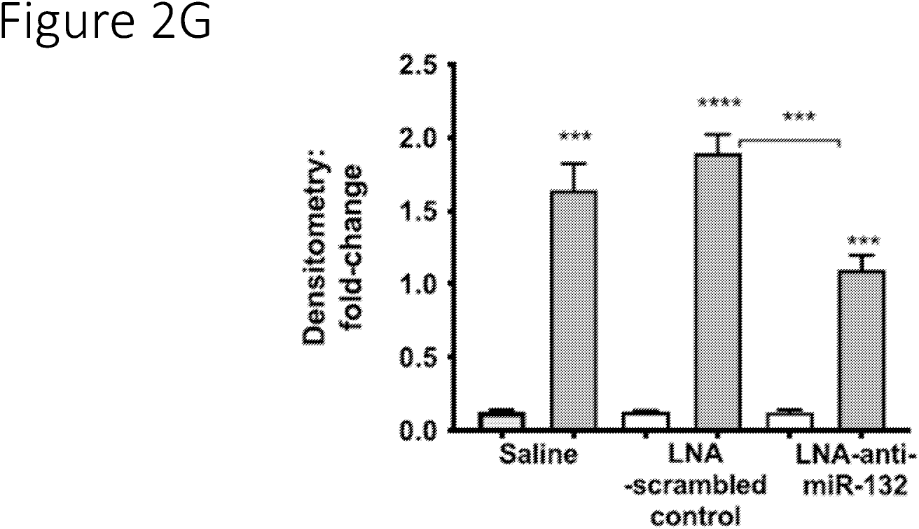

Eight-week-old male C57BL/6 wild type (WT) mice were obtained from Jackson laboratory (Bar Harbor, Maine, USA) and maintained in the animal facility. Mice were injected intraperitoneally (i.p.) either with miRNA inhibitor scrambled oligonucleotides (LNA scrambled control) or miRCURY LNA miRNA inhibitor (LNA-anti-miR-132) (15 mg/kg; Exiqon, USA) as shown in FIG. 2A (n=8 per group). Number of mice was determined based on previous reports that used in vivo delivery of LNA-anti-miRs in other disease models (Elmen et al. 2008; Bernardo et al. 2012). The inhibitors were injected 24 h prior to CCl₄ regimen every week. Throughout the experiment mice received either corn oil (vehicle) or CCl₄ (0.6 ml/kg; i.p. diluted in corn oil at 1:3 ratio) twice a week for total 2 weeks, and mice were sacrificed 72 h following final CCl₄ injection. At the end of treatment, blood was collected from mouse facial veins, and plasma was separated and stored at -80° C. for further analyses. Liver tissue was immediately either snap frozen in liquid nitrogen for protein analyses or stored in RNAlater (Qiagen, USA) for subsequent RNA analysis.

Some mice were perfused to isolate cells hepatocytes and KCs using established protocols and one lobe of the liver was dissected out before proceeding for profusion. Briefly, livers were perfused with HBSS solution containing EGTA and CaCl₂ (buffer 1) for 10 minutes followed by in vivo digestion with HBSS solution containing collagenase (Sigma Aldrich, USA) (buffer 2) for 5 minutes. Liver cells were released from perfused livers in buffer 2 by separating the liver lobes via scalpel under sterile conditions before being filtered through a cell strainer (100 pm). To separate hepatocytes from non-parenchymal cells, the cell suspension was centrifuged at 200 g for 5 minutes at room temperature. The pellet containing hepatocytes was washed 2 times with buffer 1 and lysed in Qiazol lysis buffer (Qiagen, USA). To isolate KCs, the supernatant was layered on Percoll gradient (added 25% Percoll in 50 ml tube and underlayer with 50% Prepoll) and centrifuged at 1600 g for 30 minutes. The inter-cushion layer was collected carefully and washed with PBS two times, and the resulting cells were cultured in low glucose DMEM supplemented with 10% FBS and antibiotics. The free-floating cells were removed after two sessions of 3-4 h of plating with PBS, and new medium was then added. The following day, cells were lysed in Qiazol lysis buffer (Qiagen, USA).

### MiRNA Target Analysis and TCGA Data Analysis

Experimentally validated targets of miR-132 in the liver were predicted and visualized by miRNet. UALCAN platform was used to analyze HCC tumor transcriptome miR data and plot survival information (Chandrashekar et al. 2017). TP53 mutation status was obtained from TCGA whole exome sequencing data. Mutation Annotation Format (MAF) files (VarScan2) was used for mutation calling.

Only samples with both miR expression profiling and mutation or mRNA expression profiling were considered. Correlation between miR-132 and mRNA was quantified with Pearson’s correlation coefficient, and correlation co-efficients with p value % 0.05 were considered statistically significant. The Mann- Whitney U test was used to compare expression between different groups of samples. We generated survival curves of HCC cases in the TCGA cohort according to the expression status of the miR-132, and the Kaplan-Meier curve was plotted.

### Patient Samples

Fiber tissues from control subjects and alcoholic patients with fibrosis/cirrhosis (n=8) and HCV patients with cirrhosis (n=8-10) were included in the study.

### Histopathological Analysis

Formalin- fixed liver sections were stained with hematoxylin-eosin and Sirius Red stain using standard protocol. The slides were analyzed under light microscopy at 100X and 200X.

### RNA Analysis and qPCR

10-20 mg of liver tissue was homogenized in QIAzol Lysis reagent (Qiagen, USA) using stainless steel beads via TissueLyser II (Qiagen, USA). Total RNA was extracted using the miRNeasy™ kit (Qiagen, USA) as recommended by the manufacturer (Bala et al. 2011). For mRNA analysis, cDNA was transcribed with the iScript reverse transcription system kit (BioRad, USA) and real-time quantitative polymerase chain reaction was performed via CFX96 iCycler (BioRad, USA). Quantitative analyses of genes were performed using gene-specific primers. Cq value was normalized to 18S or β actin mRNA and differential expression fold changes were calculated using the delta-delta Ct method. For miR analysis, TaqMan miR assays (Applied Biosystems, USA) were used as described earlier (Bala et al. 2011; Bala et al. 2012). SnoRNA-202 (mouse samples) or RNU48 (Human) were used to normalize the technical variations between the samples.

### Western Blot Analysis

Whole cell lysates were extracted from livers as described (Bala et al. 2016). Briefly, 10 mg of liver tissue was homogenized in RIPA buffer containing protease and phosphatase inhibitors via TissueLyser II (Qiagen, Germany). Homogenized samples were centrifuged, and resulting clear lysates were stored at -80° C. Protein mass was quantified via Bradford assay using Bio-Rad protein assay dye reagent (BioRad, USA). Equal amounts of protein (20ug) were separated in 10% SDS polyacrylamide gel, transferred to nitrocellulose membrane overnight, and blocked for 1 h in blocking buffer. Blot was incubated overnight at 4° C. in blocking buffer with primary antibodies for a smooth muscle actin (Abeam cat# ab205718) and MMP12 (Abeam, USA), and subsequently washed with IX TBST three times. For detection, anti-mouse secondary HRP-linked antibodies (Santa Cruz Biotechnology Inc., USA) were used for 1 hour at room temperature, followed by washing three times with IX TBST. The immunoreactive bands were detected by chemiluminescence using Pierce ECL western blotting substrate (Pierce Biotechnology, USA) and LAS-4000IR (Fujifilm Corp., USA). The same blot was probed with loading control antibody P-actin (Abeam, USA).

Extracellular vesicles were lysed in RIPA buffer and checked for CD63 expression by Western blot analysis as described previously (Momen-Heravi et al. 2015).

### NanoSight/Nanoparticle Tracking Analysis (NTA)

The amounts and diameters of EVs from plasma were determined using the NanoSight NS300 system (NanoSight, UK) as described (Momen-Heravi et al. 2014). NTA post-acquisition settings were kept constant for all samples, and each video was analyzed to give the mean, median, and mode vesicle size as well as concentration estimates. Each sample was measured three times. The concentration of particles (particles/ml) and size distribution (in nanometers) were evaluated using the included NTA software.

### Electron Microscopy

For electron microscopy, EVs were isolated using the ExoQuick method (System Biosciences, USA), as described earlier (Momen-Heravi and Bala 2018). Briefly, plasma was passed through a 0.8 pm filter, and ExoQuick was added as described by the manufacturer. Purified EVs were re-suspended in PBS then placed on a formvar-coated copper grid and incubated for 30 minutes as described (Momen-Heravi et al. 2015). The grid was washed with PBS 3 times and samples were fixed for 10 minutes by placing the grid onto 2% paraformaldehyde. Fixation was followed by several washes with deionized water, and samples were contrasted by adding 2% uranyl acetate for 15 minutes. Samples were embedded by adding a drop of 0.13% methyl cellulose and 0.4% uranyl acetate for 10 minutes before subsequent examination in a Philips CM 10 transmission electron microscope and imaging via Gatan CCD digital camera.

### Biochemical Analysis

Caspase 3 activity was measured from whole liver cell lysates using a caspase 3 activity assay (R&D, USA), following the manufacturer’s suggested protocol.

The liver injury was assessed at the enzymatic level by measuring alanine aminotransferase (ALT) activity from plasma samples using a kinetic method (TECO Diagnostics, CA, USA) as per manufacturer’s instructions.

### In Vitro Transfection Studies

RAW 264.7 macrophages were cultured and maintained in high-glucose DMEM (Thermo Fisher Scientific, Waltham, MA, USA) containing 10% FBS (HyClone Laboratories, Logan, UT, USA) at 37° C. in a 5% CO2 atmosphere, as described previously (Zhang et al. 2014; Chen et al. 2014), For overexpression of miR-132, cells were seeded in 24-well plates, and on the next day cells were treated either with a negative control mimic #1 or miR-132 mimic (150 pmol); for inhibition of miR-132, cells were treated either with negative control inhibitor #1 or miR-132 inhibitor (150 pmol) for 24 h (Applied Biosystems, Foster City, CA, USA) using lipofectamine RNAi max reagent (Thermo Fisher Scientific, CA, USA), as described previously (Zhang et al. 2014; Chen et al. 2014). Some cells were either treated or not with 0.1% CCl₄ for the last 24 h of the experiment, as described previously (Bala et al. 2020). Cells were washed with IX PBS two times and lysed in RNA lysis buffer for total RNA extraction or radioimmunoprecipitation assay buffer for protein extraction and stored at -80° C. for further analysis.

### Enzyme-Linked Immunosorbent Assay (ELISA) Assay

Cell lysates were used to measure IL-1β protein levels using ELISA as described by manufacturers (BioLegend, San Diego, CA, USA).

### Cell Culture

Hepal.6 mouse hepatocyte cell line was purchased from ATCC and maintained in a low-glucose DMEM (Thermo Fisher Scientific, MA, USA) containing 10% FBS (HyClone Laboratories, UT, USA) at 37° C. in a 5% CO2 atmosphere as described previously (Zhang et al. 2014; Chen et al. 2014). LX2 cells were cultured in low glucose DMEM medium as described previously4 and treated with 5 ng/ml TGFβ for indicated times. Cells were washed with 1XPBS twice and cells were lysed in Qiazol (Qiagen, USA) and processed for total RNA extraction (Zymo Research, USA).

### Transfection

For overexpression of miR-132, cells (macrophages and hepatocytes) were seeded onto 24-well plates and next day, cells were treated either with a negative control mimic #1 or miR-132 mimic (150 pmol) and for inhibition of miR-132, cells were treated either with negative control inhibitor #1 or miR-132 inhibitor (150 pmol) for 24 h (Applied Biosystems, CA, USA) using lipofectamine RNAi max reagent (Thermo Fisher Scientific, CA, USA) as described previously (Zhang et al. 2014; Chen et al 2014). Some cells were either treated or not with 0.1% CCl₄ for the last 6 h of experiment as described previously (Bala et al. 2020). Cells were washed with 1XPBS for two times and lysed in RNA lysis buffer for total RNA extraction or RIPA buffer for protein extraction and stored at -80° C. for further analysis.

### Statistical Analysis

Statistical significance was determined using the non-parametric Mann-Whitney U test or two tailed student T test for pair- wise comparisons based on the underlying data distribution. Non-parametric Kruskal Wallis or one way analysis of variance (ANOVA) was used for comparison between more than two groups. Data is presented as mean+/-standard error and considered statistically significant at p<0.05.

### Example 2- Induction of miR-132 in the Fibrotic Livers of Alcoholic Patients and in CC1₄-Induced Liver Fibrosis Mouse Model

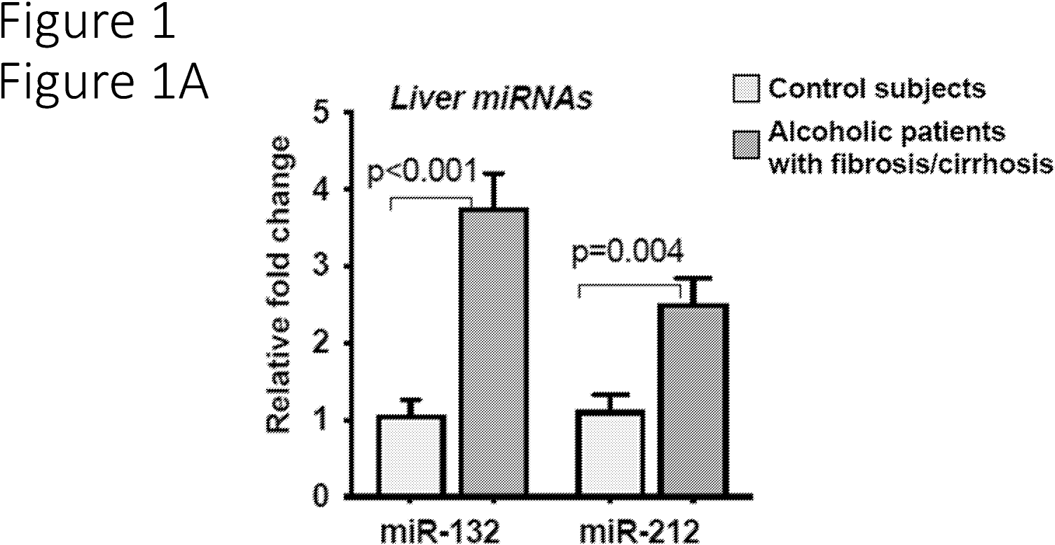

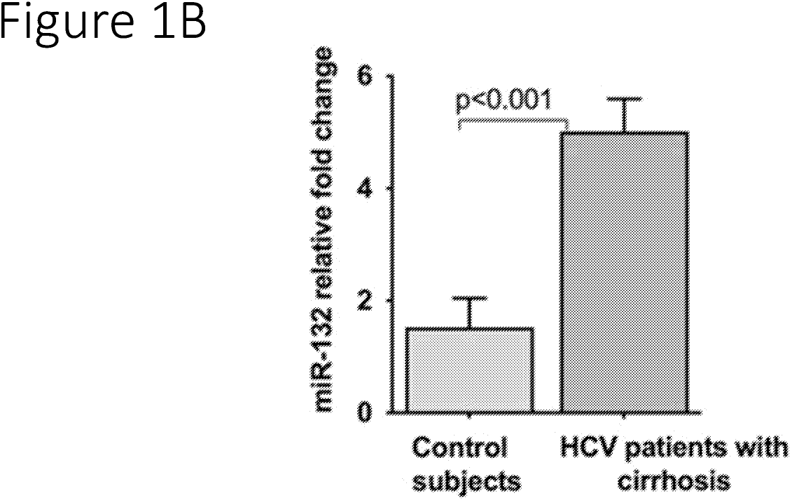

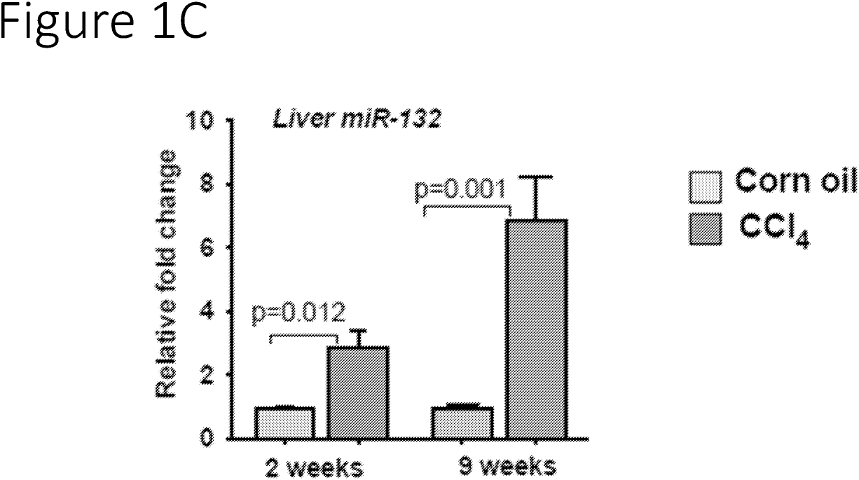

Previously, it was shown induction of miR-132 in the liver, isolated KCs, and hepatocytes after chronic alcohol feeding in mice (Bala et al. 2012). Chronic liver injury caused by dietary intake (either alcohol or NASH) or viral infections can lead to downstream fibrosis and cirrhosis (Gai and Bataller 2011), therefore the levels of miR-132 in the livers of alcoholic patients with fibrosis/cirrhosis were checked. A significant induction in miR-132 expression was found in the livers of alcoholic patients with fibrosis/cirrhosis, as seen in FIG. 1A. Since miR-132 and miR-212 are closely correlated and encoded in the same intron of a small noncoding gene, miR-212 levels in these samples were also examined. Although both miR-132 and miR-212 were significantly increased in patients with alcoholic cirrhosis (p<0.05), consistent with previous reports (Wanet et al. 2012), a higher induction of miR-132 was found as compared to miR-212 (~2.5 fold) (FIG. 1A). Additionally, a significant increase in miR-132 levels was found in the cirrhotic livers of HCV patients (FIG. 1B). Therefore, the mechanistic studies were focused on miR-132.

Next, the levels of miR-132 in a CCl₄-induced liver fibrosis mouse model were checked. A significant and sustained induction of miR-132 in the livers of mice after administration of CCl₄ was found for both 2 and 9 weeks (FIG. 1C).

Collectively, the results from both human and mouse studies showed a potential role of miR-132 as a mediator of liver fibrosis.

### Example 3- Inhibition of miR-132 With LNA-Anti-miR-132 Attenuated CCl₄-Induced Liver Fibrosis

To determine the role of miR-132 in liver fibrosis, a mouse model of CCl₄-induced liver fibrosis was used. Currently, there is no widely accepted mouse model of alcoholic liver disease (ALD) that represents the liver fibrosis/cirrhosis observed in alcoholic patients. The CCl₄-induced liver fibrosis mouse model is widely used and has proven to be robust and reproducible, able to mimic the histological, biochemical, and molecular changes associated with the development of fibrosis (Iredale 2007). miR-132 function in mice was inhibited using miRCURY LNA inhibitor (LNA-anti-miR-132). The schematic of LNA-anti-miR-132 treatment schedule and CCl₄ administration is shown in FIG. 2A.

CCl₄ treatment increased the hepatic miR-132 levels in saline or scrambled control treated mice, and miR-132 was almost undetected in the treatment group (LNA-anti-miR-132) across both oil and CCl₄-treated mice (FIG. 2B). The CCl₄-induced increase in miR-212 was prevented in mice treated with miR-132 inhibitor (FIG. 2C). Consistently, this effect was specific to LNA-anti-miR-132 and was not observed in LNA-scrambled control. Histological evaluation showed that administration of LNA-anti-miR-132 attenuated the fibrosis phenotype of liver tissue in CCl₄ liver injury, as characterized by less fibrous tissue compared to the controls (FIG. 2D). The liver damage assessed by histological evaluation of H&E staining revealed fewer mononuclear cells (results not shown) and decreased ALT levels in the livers of LNA-anti-miR-132-treated mice after CCl₄ treatment (FIG. 2E). CCl₄ treatment was found to have induced the expression of fibrogenic gene transcripts (collagen la, TIMP1 and TGFβ) in LNA-scrambled control-treated mice, whereas LNA-anti-miR-132 treatment attenuated CCl₄-induced increase in collagen la and inhibited the induction of TIMP1 and TGFβ transcripts (FIG. 2F).

These findings were further corroborated via decreased smooth muscle actin protein levels in the livers of LNA-anti-miR-132-treated mice as compared with the LNA-scrambled control-treated mice and non-treated (saline) mice in CCl₄-induced liver injury (FIG. 2G).

### Example 4- LNA-Anti-miR-132 Attenuated CCl₄-Induced Release of Extracellular Vesicles and Caspase 3 Activity

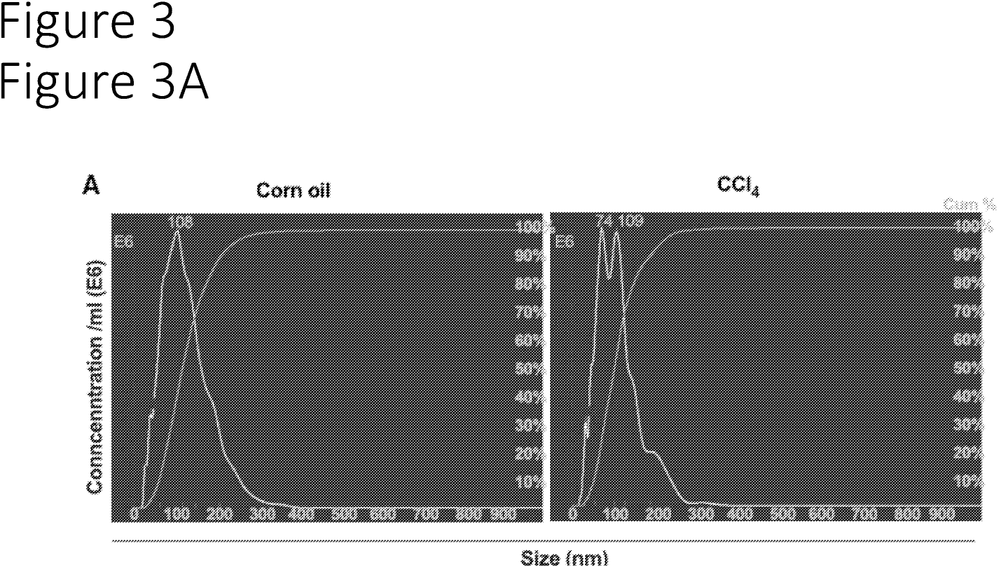

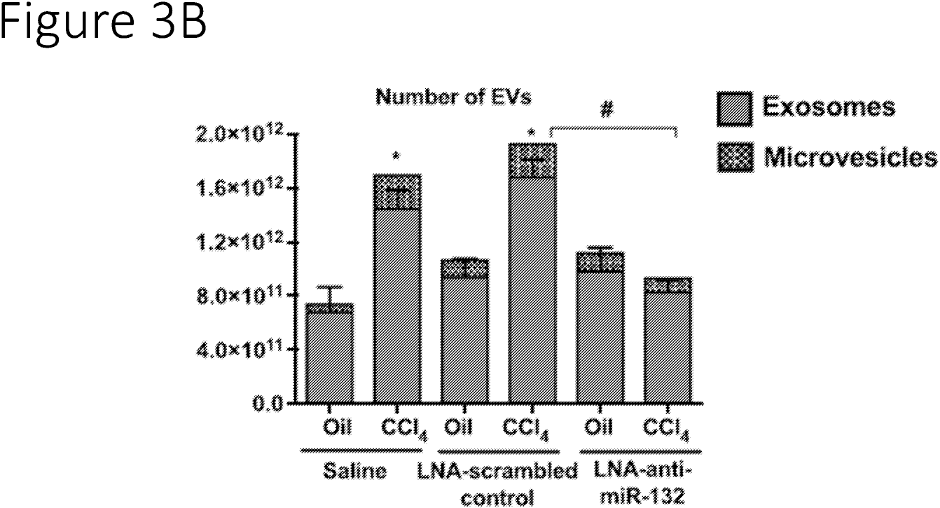

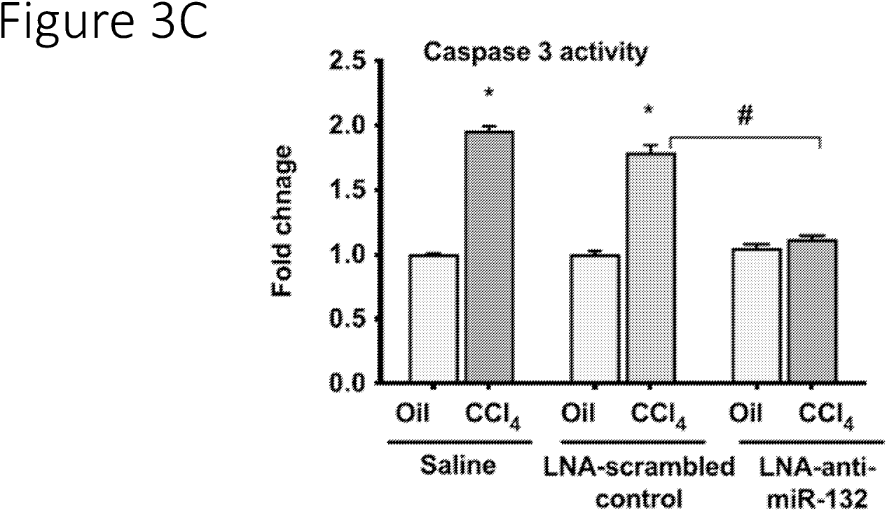

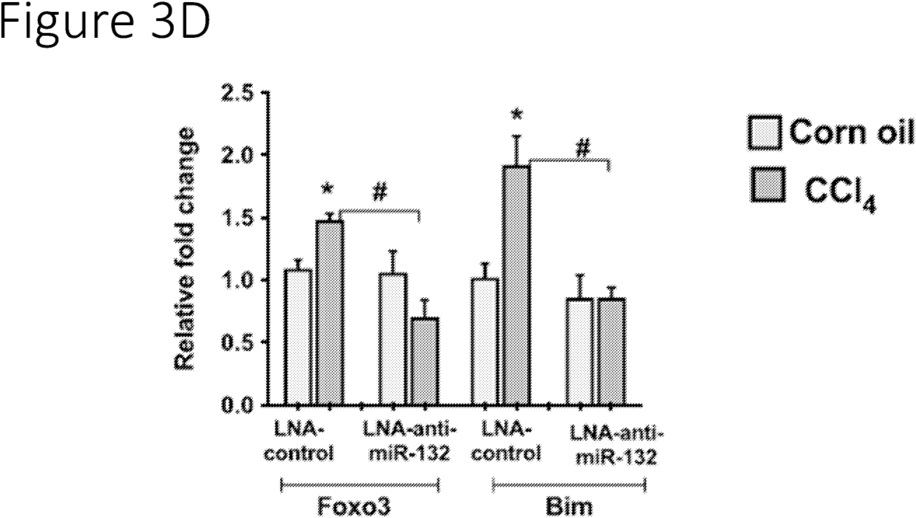

It has been well established that upon induction of cell stress, cells produce EVs (Ranghino et al. 2015; Royo and Falcon-Perez 2012), and here, consistent with those findings, increase in the total number of EVs produced was observed, which ranged from exosomes (<200 nm) to larger microvesicles (>200 nm) (FIG. 3A). The mean diameters of EVs were 320 nm for CCl₄-treated mice and 117 nm for oil-treated mice (FIG. 3A). The isolated EVs were visualized with electron microscopy and showed the morphology of extracellular vesicles described before (Momen-Heravi et al. 2014) (results not shown). Administration of LNA-anti-miR-132 significantly reduced production of EVs in CCl₄-treated mice as compared to LNA-scrambled control as well as restored the production of exosomes to the level of baseline oil-treated mice (FIG. 3B).

Caspase 3 is shown to play an important role in the promotion of fibrosis and associated cell damage as well as the production of apoptotic EVs (Lapante et al. 2009). Thus, it was hypothesized that LNA-anti-miR-132 can exert a protective effect on caspase 3 activity. Interestingly, CCl₄-induced caspase 3 activity was significantly reduced after administration of LNA-anti-miR-132 but not LNA-scrambled control, which indicated a role of miR-132 in cellular damage (FIG. 3C).

The pro-apoptotic genes Foxo3 and Bim were induced in LNA scrambled control CC1₄-treated mice, whereas increases in these genes were inhibited after administration of LNA-anti-miR-132 (FIG. 3D).

### Example 5- LNA-Anti-miR-132 Activated Anti-Fibrotic Transcripts and Suppressed Proapoptotic Mediator

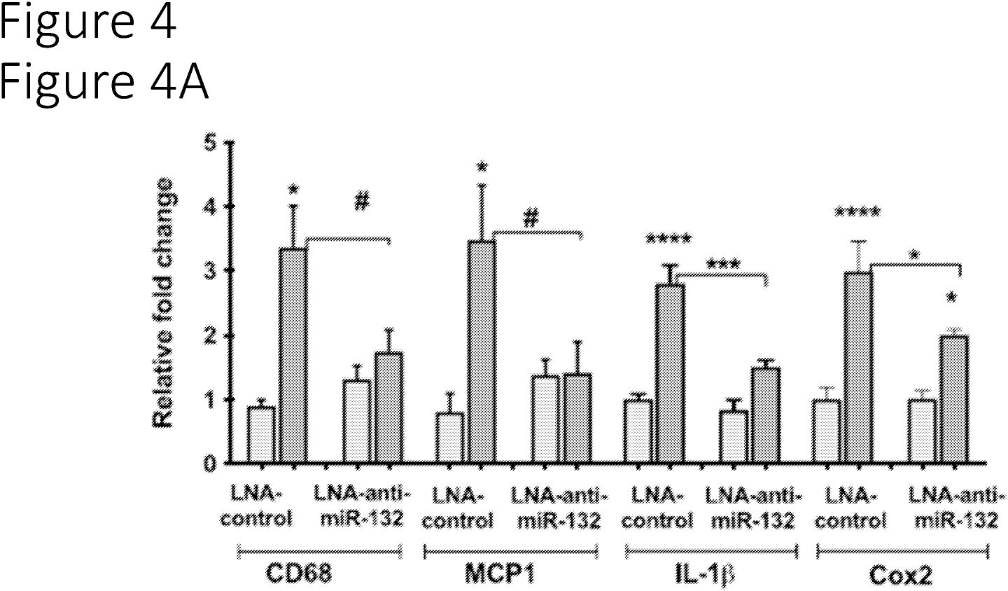

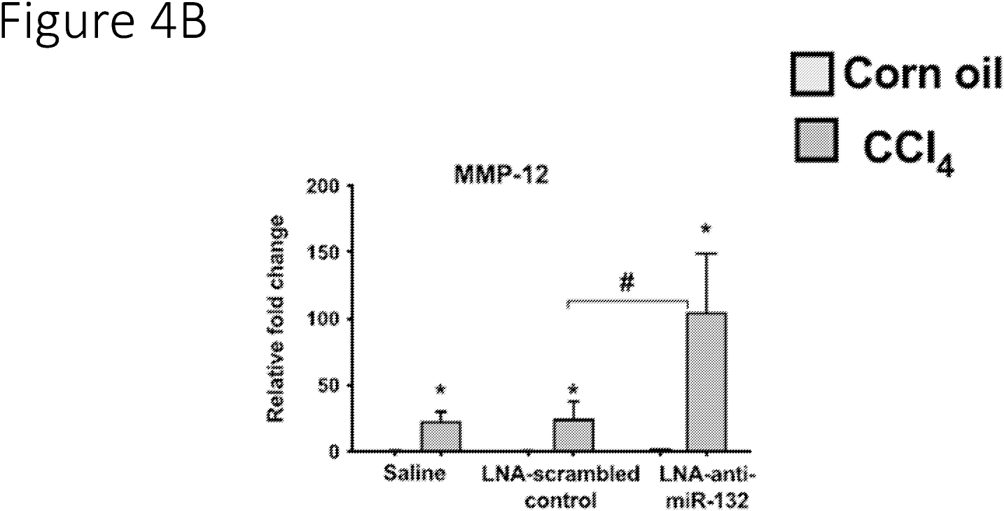

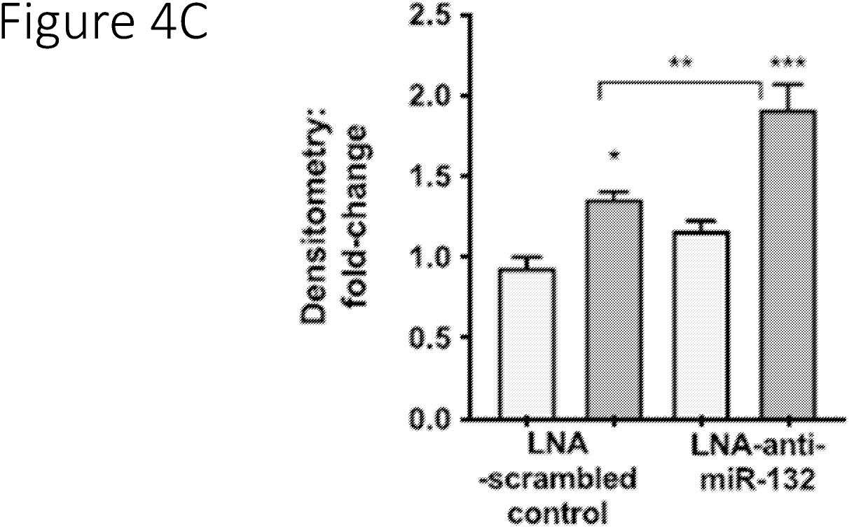

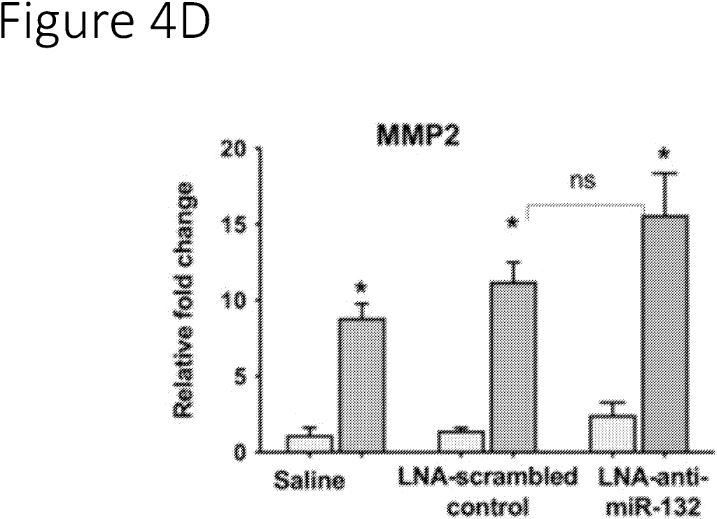

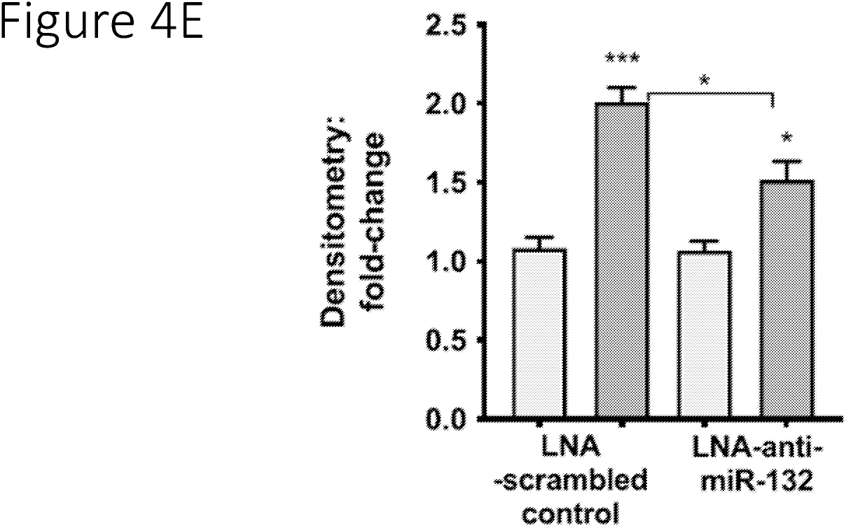

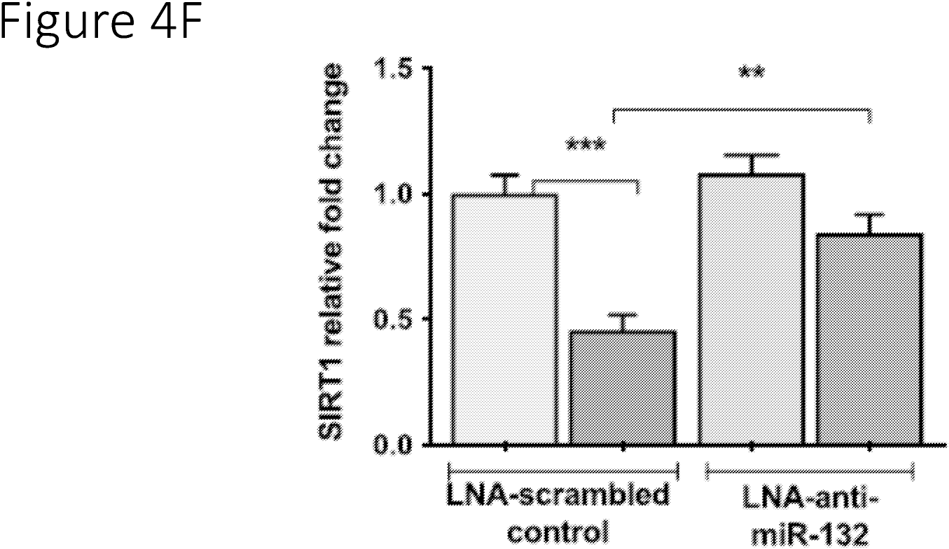

The inflammatory responses play an important role in fibrogenesis, as persistent inflammation primes fibrotic events. An increase in CD68, a macrophage inflammatory marker, and MCP1 chemokine was found in LNA-scrambled control mice, and LNA-anti-miR-132 treatment prevented an induction in these genes after CCl₄ treatment (FIG. 4A). Further, expression of inflammatory mediators IL-1β and Cox2 was induced in LNA-scrambled CCl₄-treated mice, and an attenuation in these genes was observed after LNA-anti-miR-132 treatment (FIG. 4A).

Matrix metalloproteases (MMP) are shown to play a key role in repairing connective tissue damage. Macrophage metalloelastase-12 (MMP- 12) plays an active role in the turnover of elastin (37, 38) and substantially increased levels of MMP- 12 transcripts were found in the liver after CCl₄ treatment (FIG. 4B). Furthermore, mice treated with LNA-anti-miR-132 showed a significantly higher induction of MMP-12 compared to mice treated with LNA-scrambled control after CCl₄ treatment (p<0.05) (FIG. 4B). Consistently, the active forms of MMP-12 were increased to a greater extent in the livers of mice treated with LNA-anti-miR-132 as compared to mice treated with LNA-scrambled control after CCl₄ treatment (FIG. 4C). Enhanced levels of MMP2 transcripts in LNA-anti-miR-132- CCl₄-treated mice was also found (FIG. 4D).

Further, protein levels of vimentin, an inducer of EMT process, were attenuated in LNA-anti-miR-132-treated mice compared to LNA-scrambled controls after CCl₄ treatment (FIG. 4E). SIRT1, a validated miR-132 target, is involved in inflammatory and fibrotic pathways, and decreased expression of SIRT1 was reported in liver fibrosis. The results indicated a decrease in SIRT1 expression after CCl₄ treatment in LNA-scrambled mice, and LNA-anti-miR132 treatment rescued the reduction in SIRTI (FIG. 4F).

### Example 6- Induction of miR-132 Expression in Kupffer Cells and Hepatocytes After CCl₄ Treatment

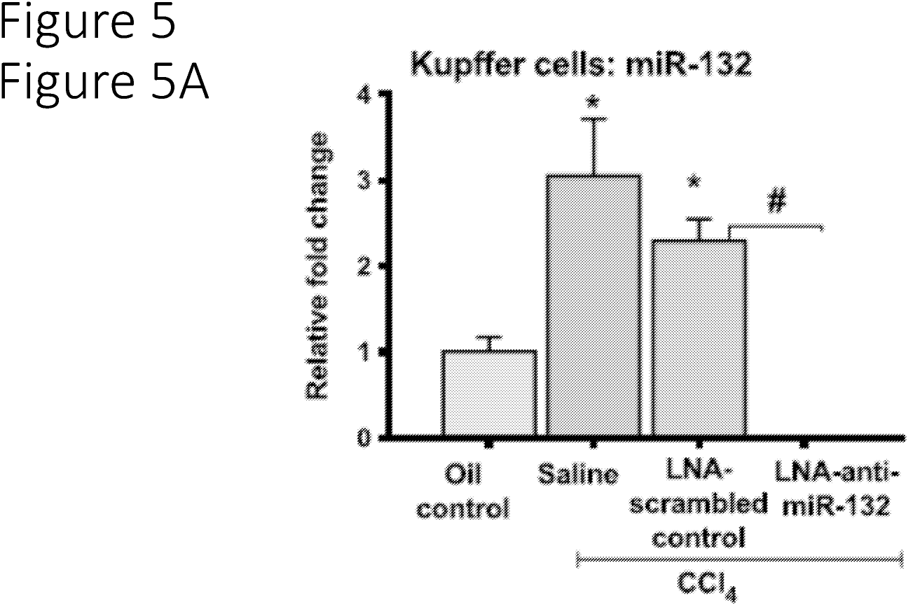

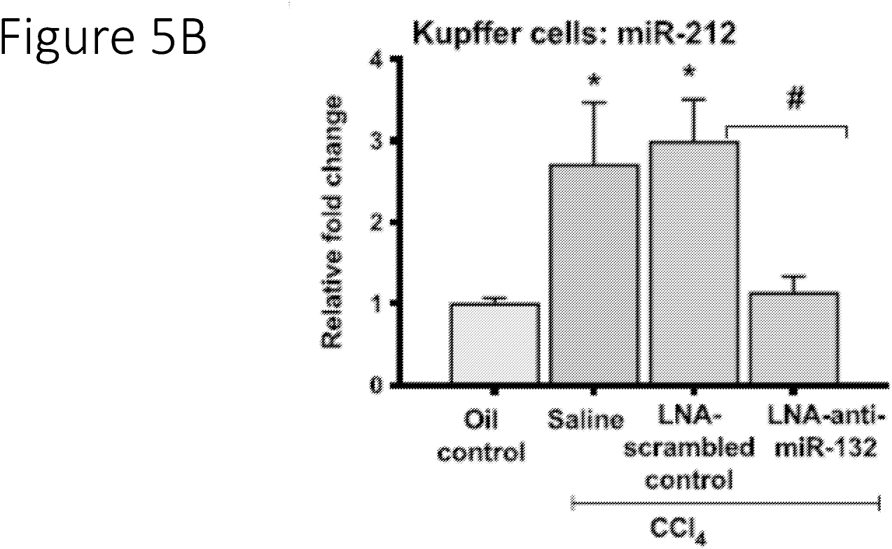

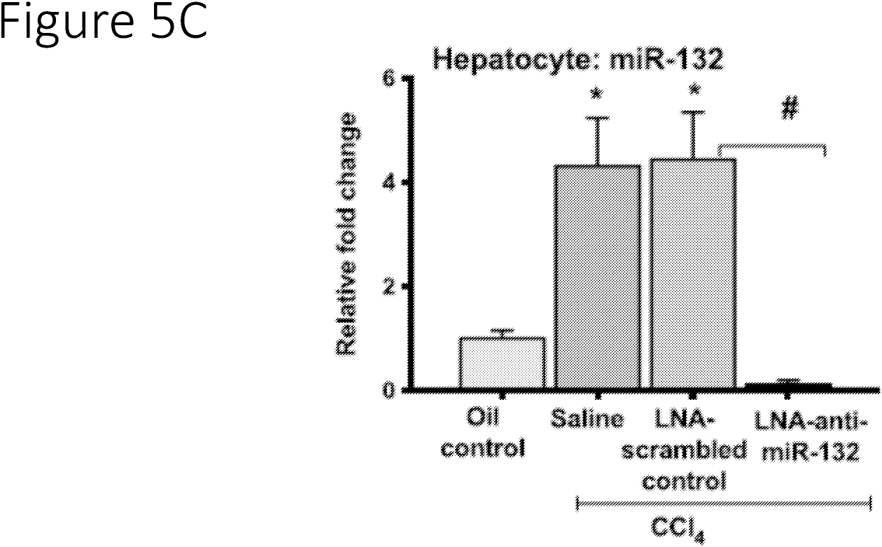

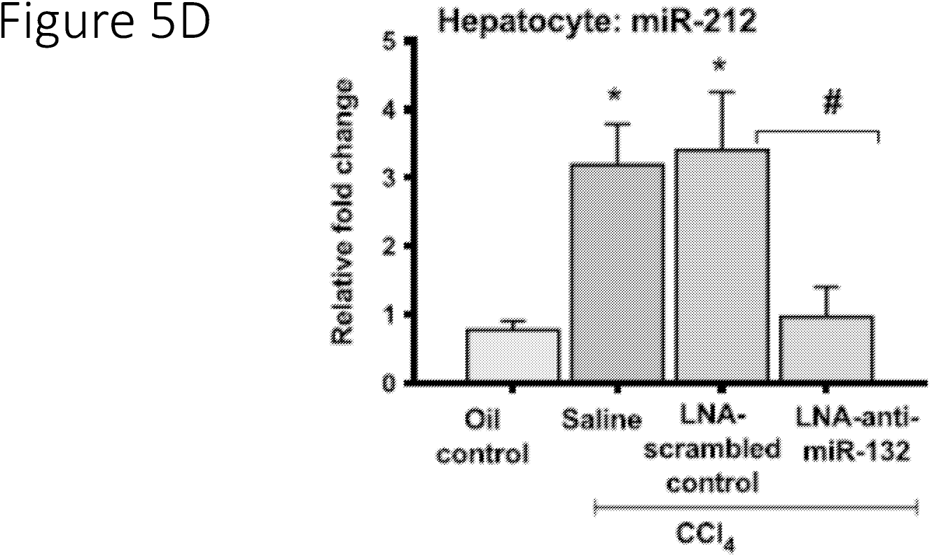

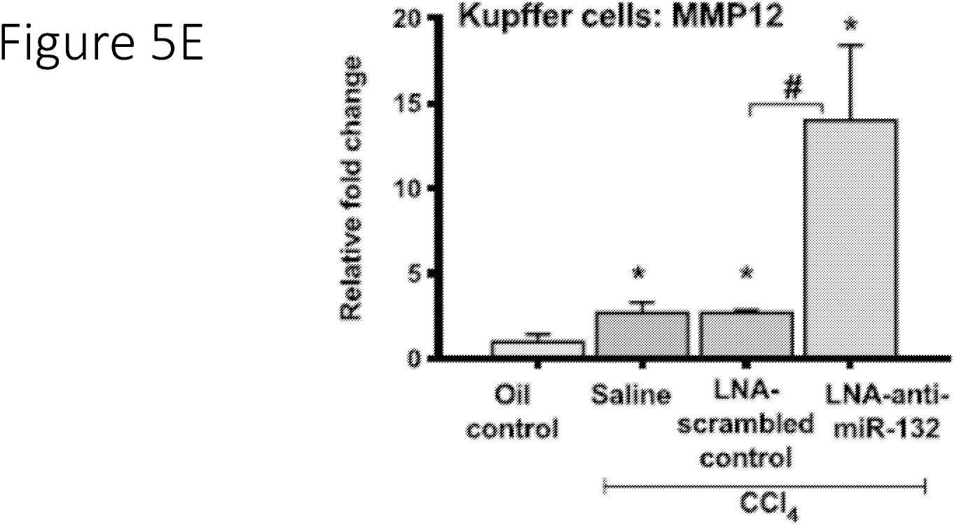

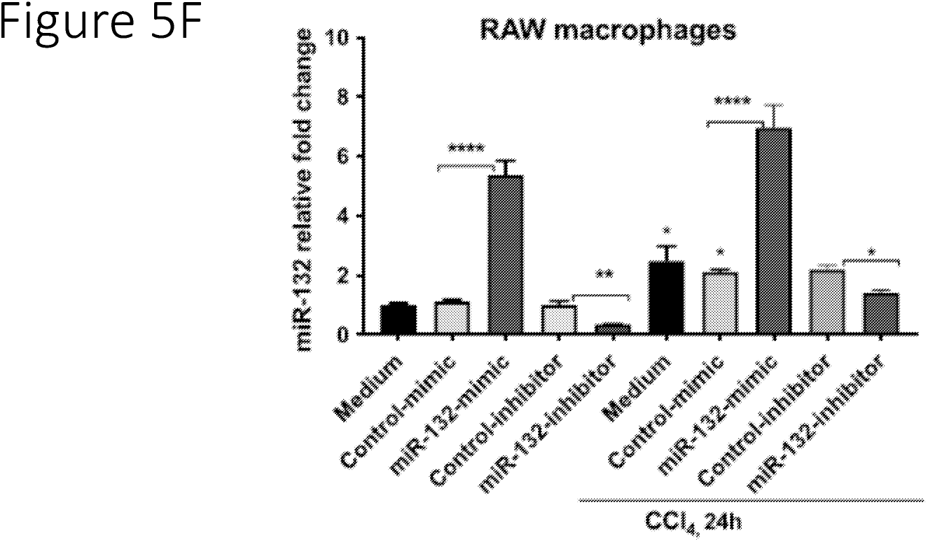

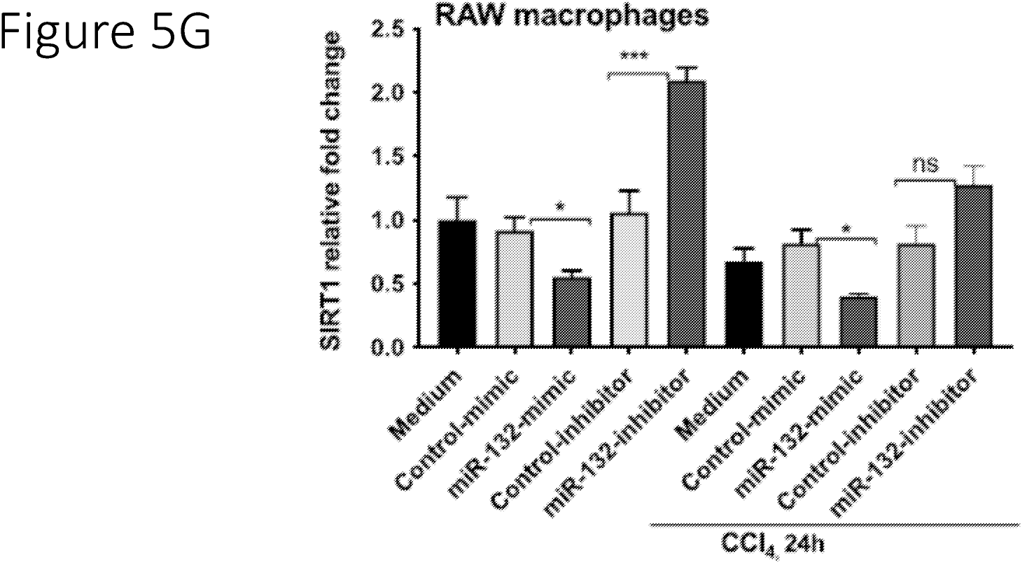

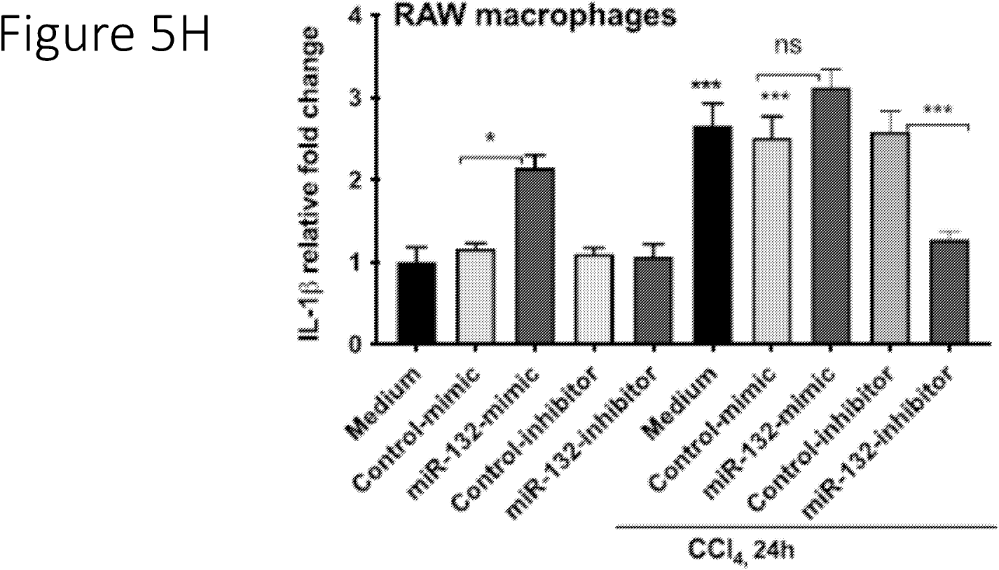

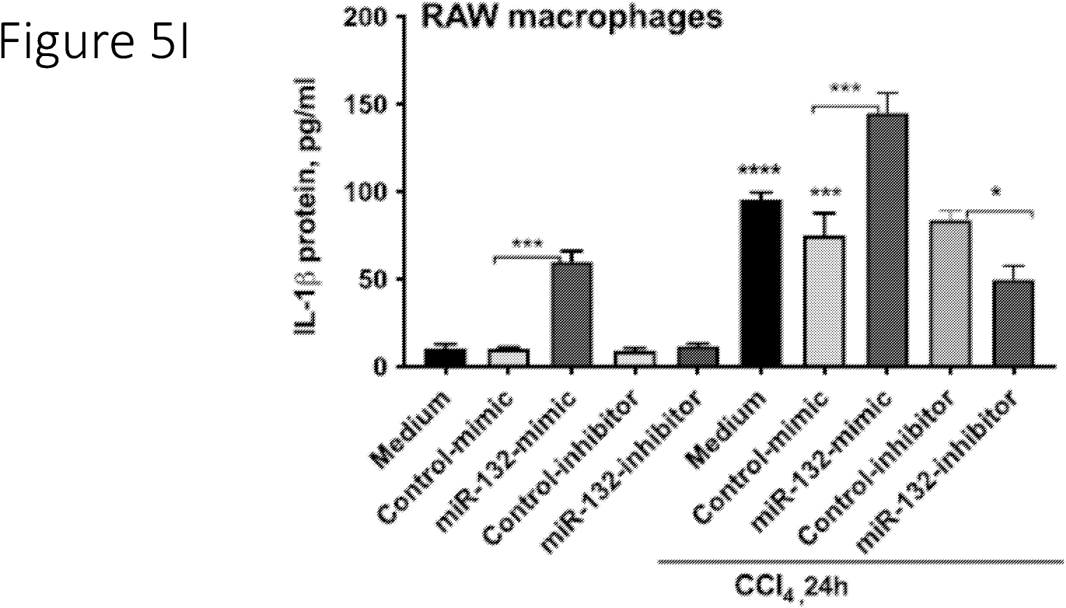

To determine the cellular source of the hepatic miR-132 upregulation, KCs and hepatocytes were isolated after 2 weeks of CCl₄ treatment. The results indicated an increase of miR-132 (FIG. 5A) and miR-212 (FIG. 5B) in isolated KCs after CCl₄ administration. LNA-anti-miR-132 treatment significantly reduced miR-132 levels in KCs (FIG. 5A), whereas miR-212 expression was found to have decreased to that of oil-treated mice (FIG. 5B). Consistently, levels of miR-132 were significantly increased in hepatocytes after CCl₄ challenge, and administration of LNA-anti-miR-132 decreased levels of miR-132 more than 20 fold (FIG. 5C). CCl₄-induced increase in miR-212 was prevented in hepatocytes isolated from LNA-anti-miR-132-treated mice, suggesting a master regulatory role of miR-132 in liver injury (FIG. 5D).

Upon discovering augmentation of MMP12 in the livers of mice treated with LNA-anti-miR-132, MMP12 expression in isolated KCs and hepatocytes was investigated. The results indicated an increase in MMP12 in isolated KCs after CCl₄ treatment (FIG. 5E). CCl₄-induced increase in MMP12 was further amplified in KCs isolated from LNA-anti-miR-132-treated mice (FIG. 5E).

To determine the mechanistic role of miR-132, in vitro studies were performed using RAW 264.7 mouse macrophages. An induction in miR-132 expression was observed in cells -treated with 0.01% CCl₄, and, regardless of CCl₄ treatment, miR-132 levels were induced in the presence of miR-132 mimic and reduced in the presence of miR-132 inhibitor (FIG. 5F). Similarly, regardless of CCl₄ treatment, SIRT1 levels were shown to vary indirectly with miR-132 expression (FIG. 5G). miR-132 overexpression increased IL-1βb mRNA (FIG. 5H) and protein levels (FIG. 5I) compared to negative control mimic-treated cells with or without CCl₄ treatment. A decrease in IL-1β mRNA (FIG. 5H) and protein levels (FIG. 5I) was found in cells treated with miR-132 inhibitor compared to negative control inhibitor- treated cells after CCl₄ treatment.

Similar findings were observed for TGFβ, wherein miR-132 mimic induced TGFβ levels (FIGS. 5J-5M).

Further, in Hepal.6 mouse hepatocytes, miR-132 overexpression resulted in increases in TGFβ, vimentin, and N-cadherin expression (FIGS. 5N-5O.) No cellular toxicity after CCl₄ treatment was found.

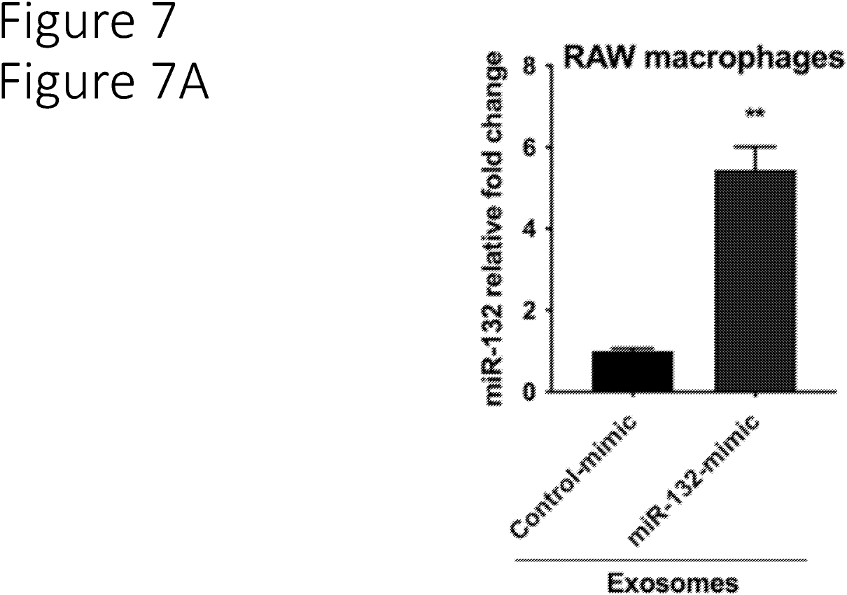

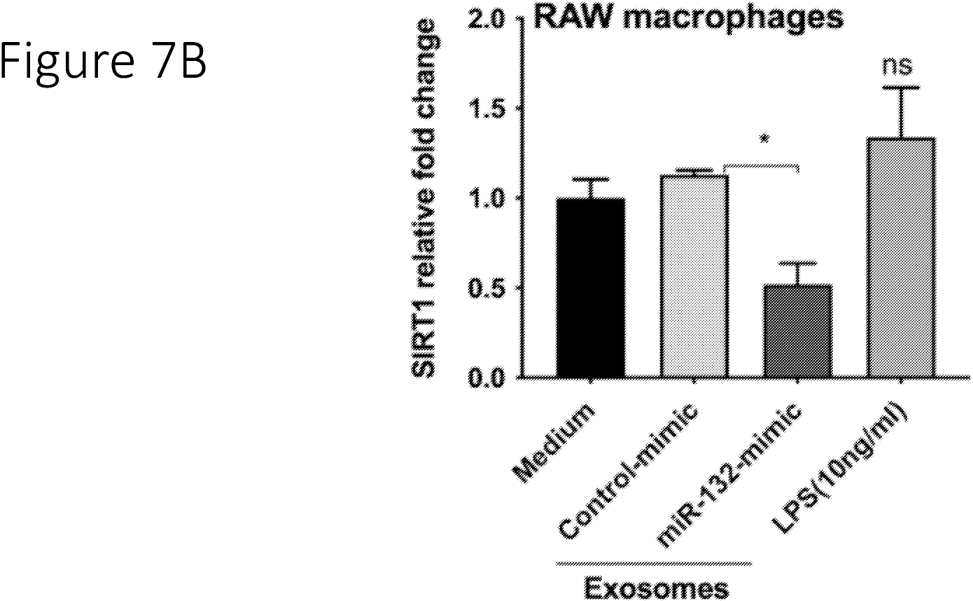

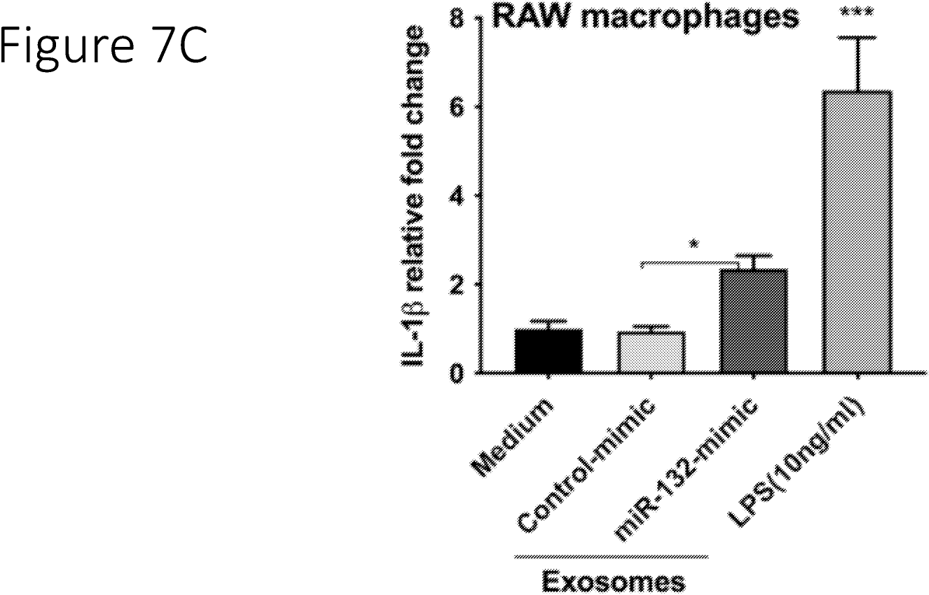

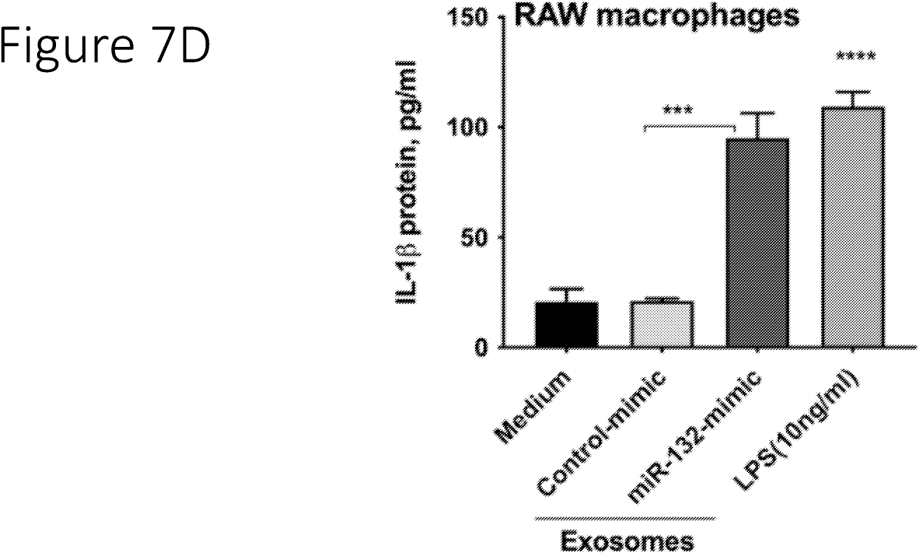

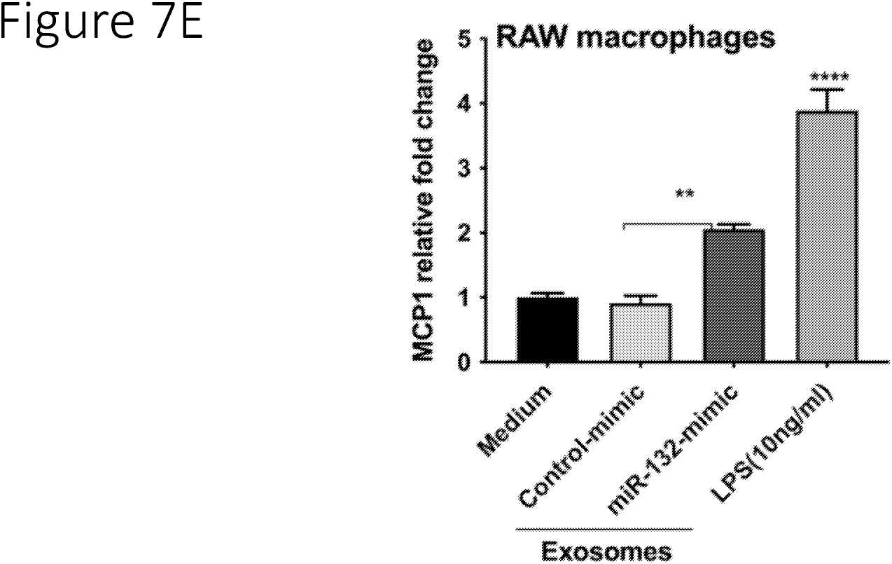

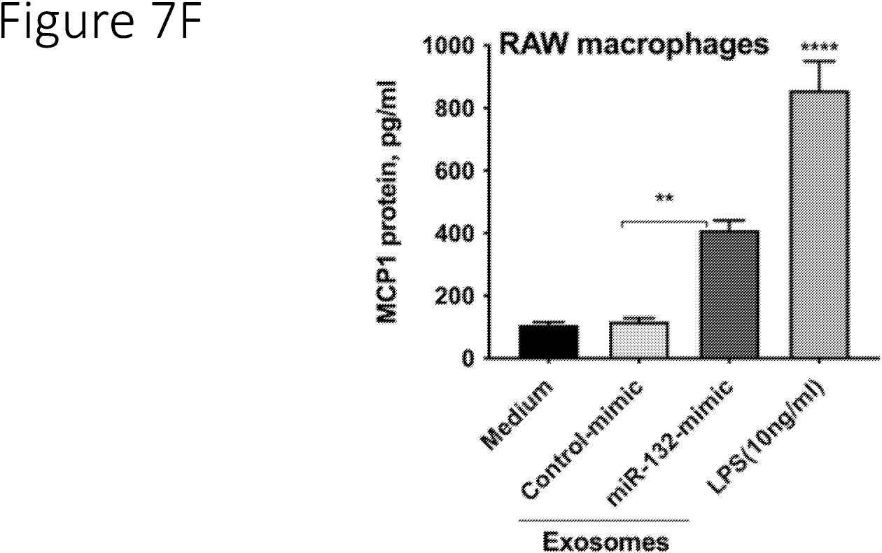

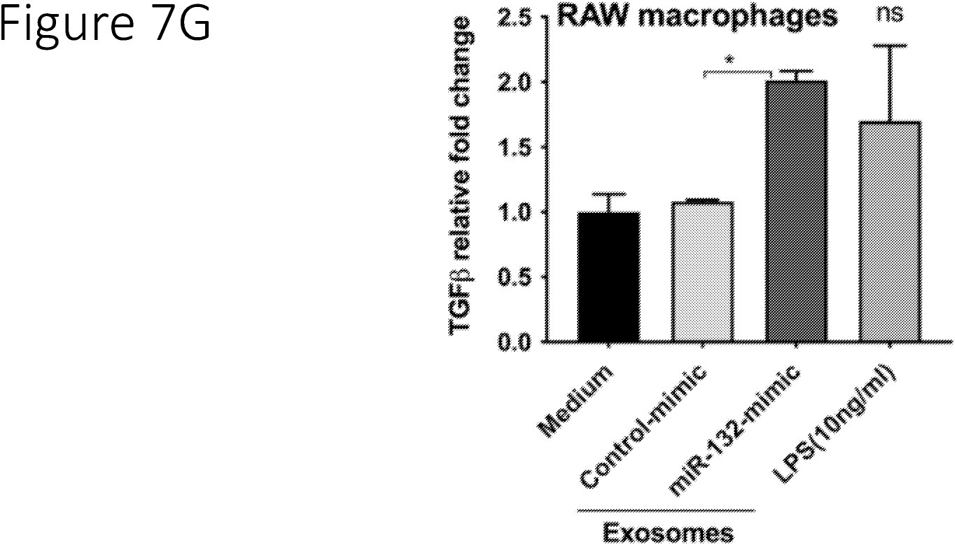

The findings of miR-132 regulating SIRT1 were further confirmed by simulation experiments, wherein miR-132 mimic-loaded exosomes were cultured with naive RAW macrophages. A reduction in SIRT1 expression was found in cells treated with miR-132 mimic-loaded exosomes compared to control mimic-loaded exosomes. Conversely, an induction in IL-1β (mRNA and protein), MCP1 (mRNA and protein), and TGFβ mRNA was found in cells treated with miR-132 mimic-loaded exosomes. See FIG. 7.

### Example 7 - miR-132 Targeted Cancer Related Genes and Was Upregulated in Hepatocellular Carcinoma

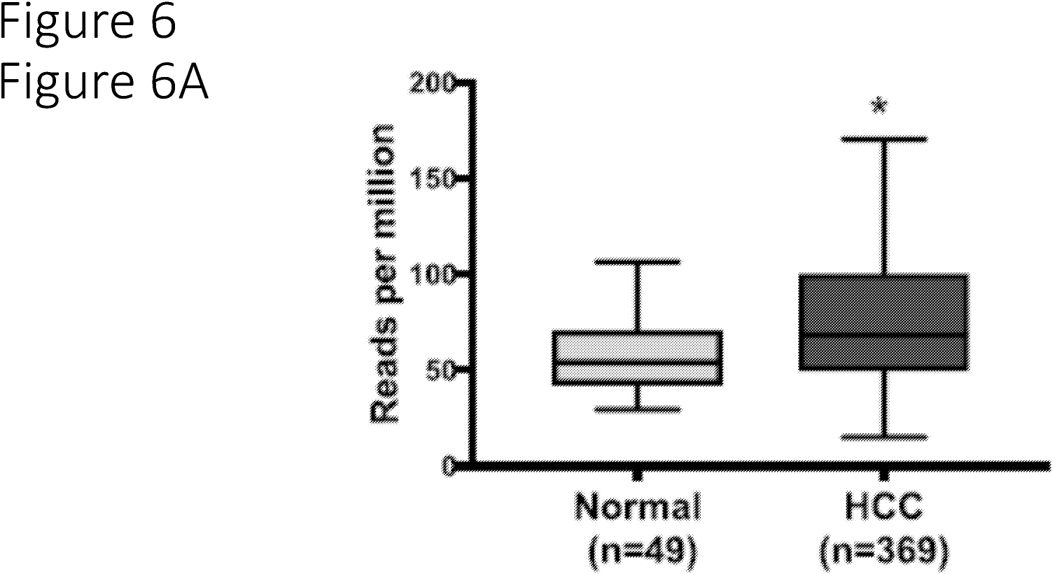

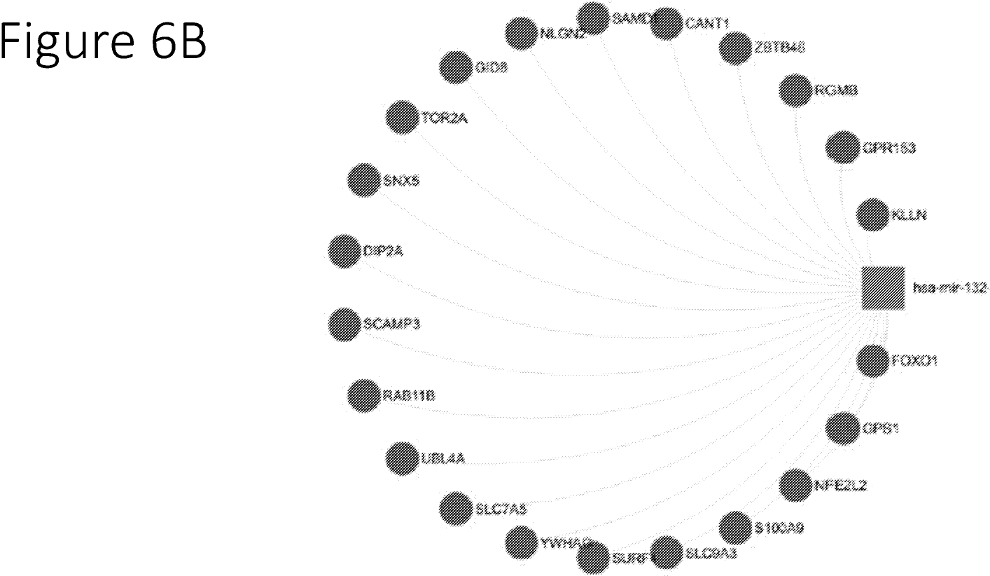

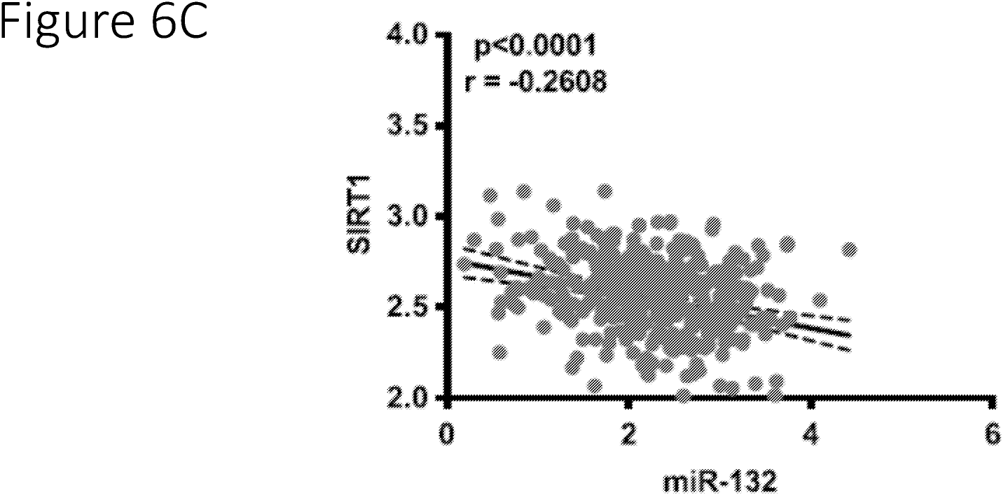

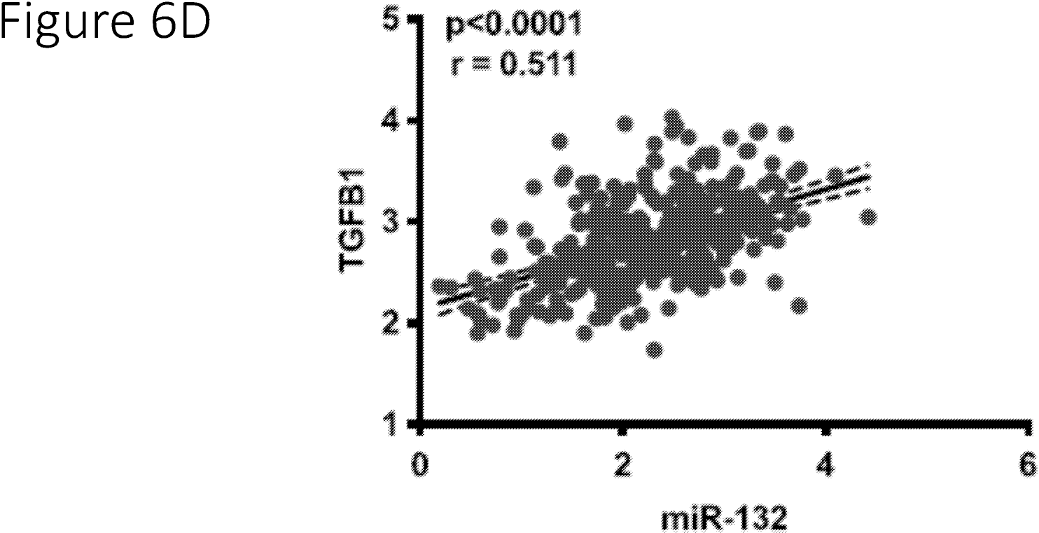

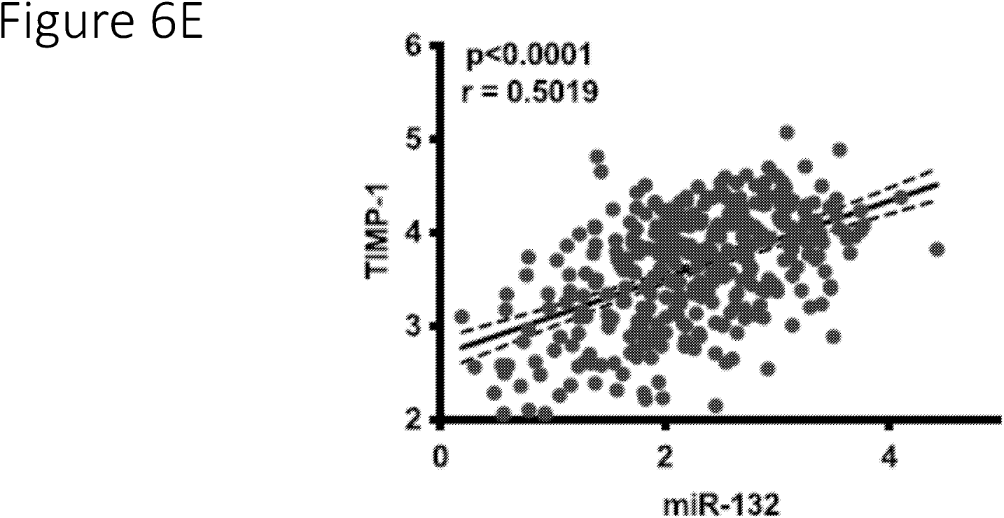

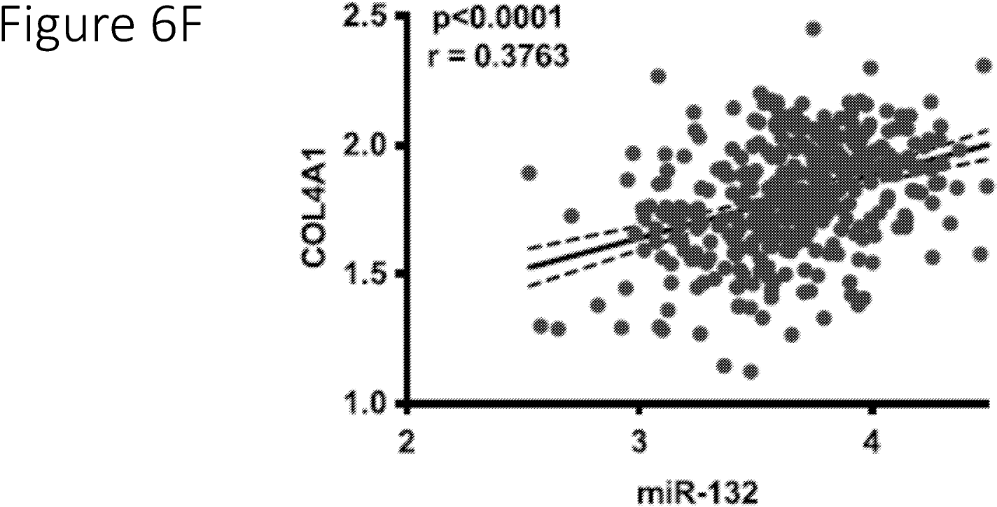

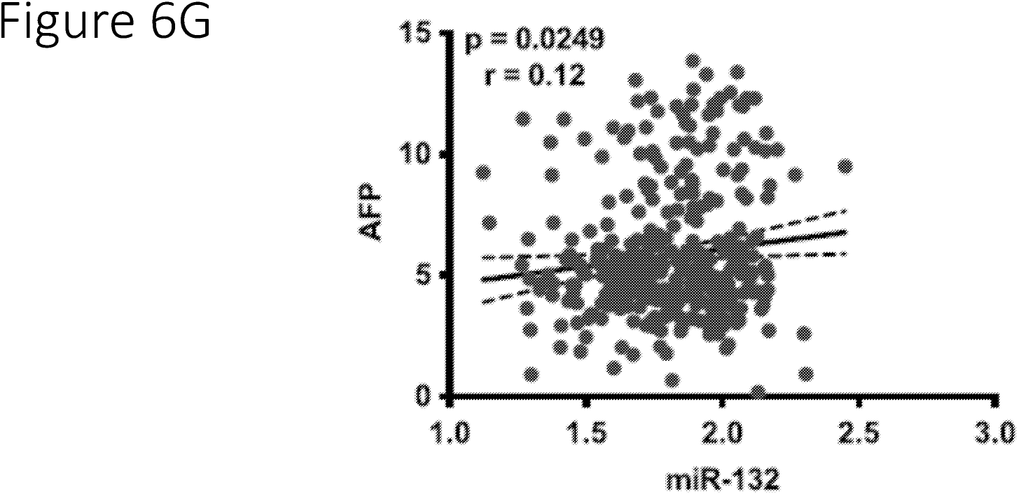

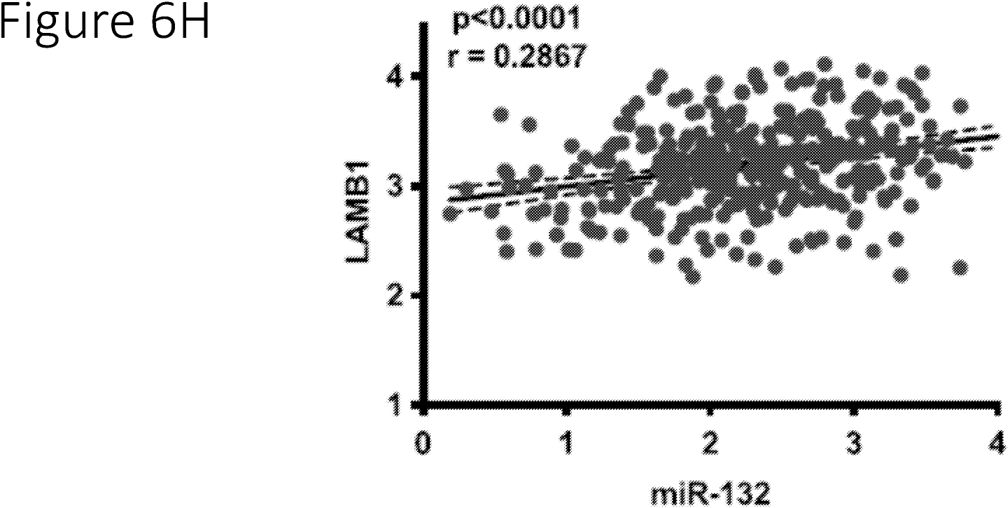

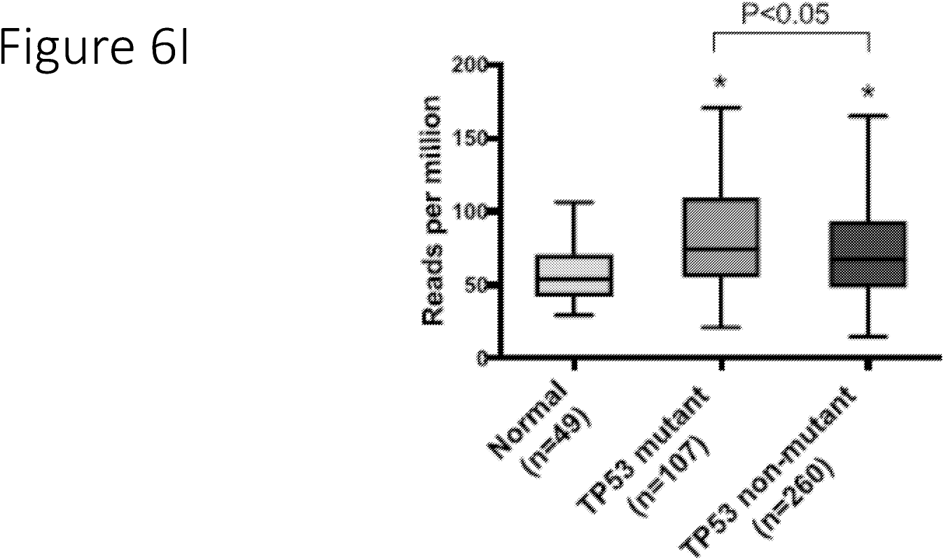

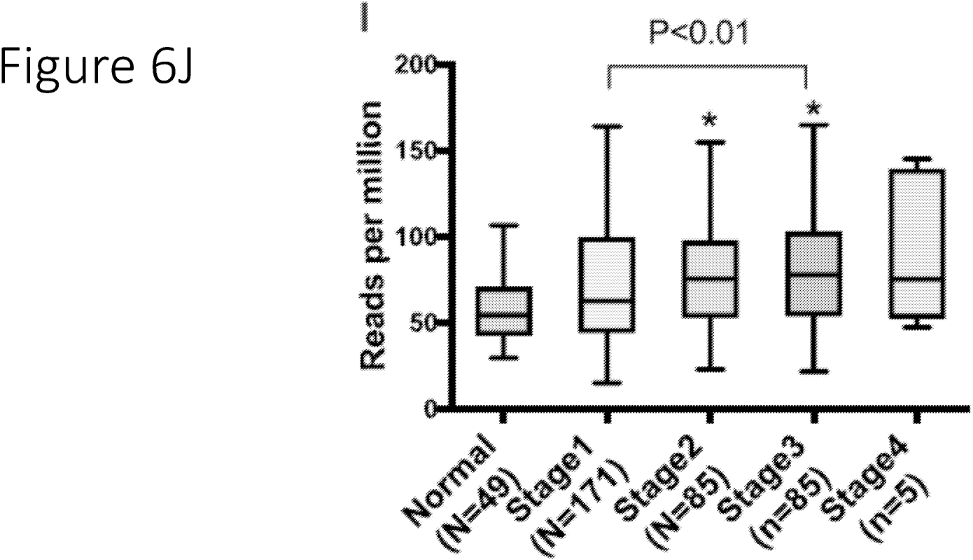

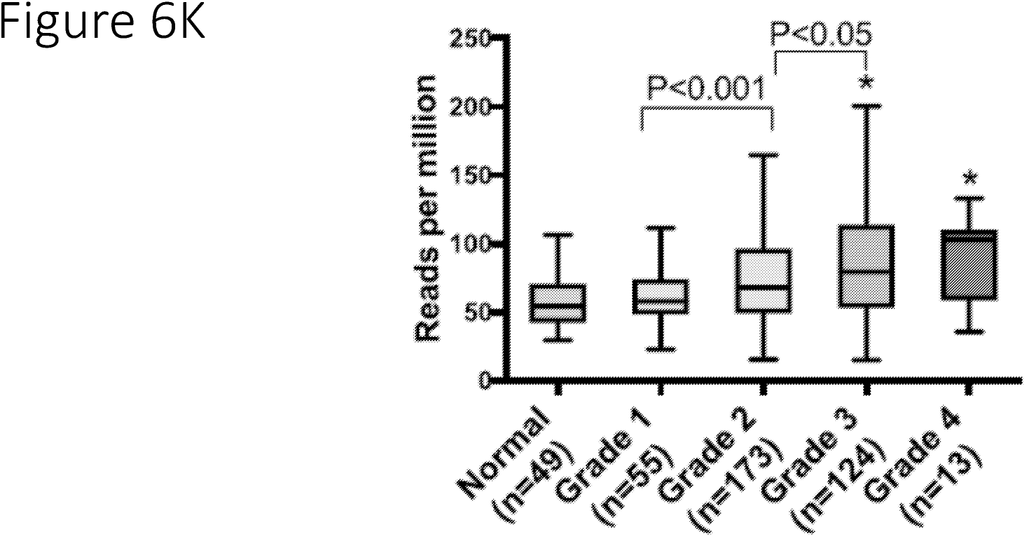

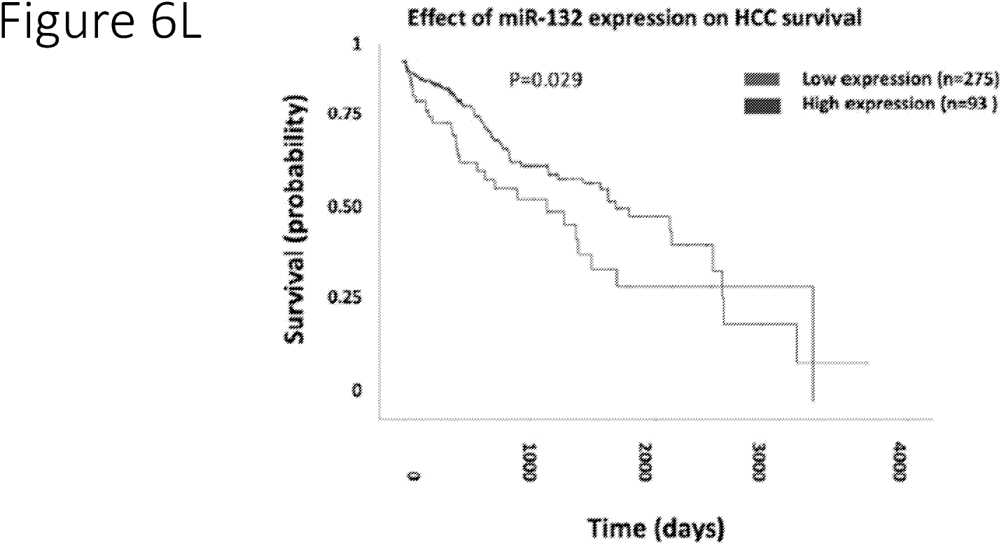

As liver fibrosis is one of the risk factors for hepatocellular carcinoma (Baffy et al. 2012), the expression of miR-132 in hepatocellular carcinoma in the tumor cancer genome atlas (TCGA) data was checked. Higher expression of miR-132-5p (miR-132) in HCC tumor tissue was found compared to the controls (p<0.05) (FIG. 6A). miR target analysis was performed to identify mRNAs that are targeted by miR-132. The analysis showed important mediators in carcinogenesis and hepatocellular carcinoma (HCC) pathogenesis in many of the targets including tumor suppressor FOXO1, CANT1, and UBL4A (FIG. 6B).

An association between miR-132 and fibrogenic genes was performed in the TCGA data, and the analysis revelated miR-132 expression was inversely correlated with SIRT1 levels (FIG. 6C) and positively correlated with TGFβ (FIG. 6D), TMIP1 (FIG. 6E), COL41α (FIG. 6F), AFP (FIG. 6G), and LAMB1 (FIG. 6H) expression. TCGA analysis also revealed an inverse correlation between SIRT1 and several fibrotic genes, such as TGFβ, CALM2, and TAF10 (results not shown).

Higher expression of miR-132 was associated with TP53 mutation in HCC (FIG. 6I) and was also associated with higher tumor grade and higher stage (FIGS. 6J and 6K). High expression of miR-132 in HCC patients was associated with significantly lower survival compared to patients with low level of miR-132 (p=0.029) (FIG. 6L).

### Example 8 - Production and Delivery of Exosomes With miR-132 Mimic as Cargo

Loading of control or miR-132 mimic into the exosomes, isolated from THP1 cells, was performed using our previously optimized protocol (Momen-Heravi et al. 2015; Momen-Heravi et al. 2014).

Briefly, exosomes were diluted in Gene Pulser® electroporation buffer (Bio-Rad Laboratories, Berkeley, CA) in 1:1 ratio and miR-132 mimic or negative control mimic (Ambion, Grand Island, NY) at 300 pmol were mixed with exosome suspension containing 1 µg/µl exosomal protein. The suspension was transferred into cold 0.2 cm electroporation cuvettes and electroporated at 150 kV and 100 µF using a Gene pulser II System (Bio-Rad Laboratories, Berkeley, CA) for electroporation. The exosomes were treated with one unit of RNase H to eliminate free-floating miR and re-isolated using ExoQuick-TC™. These loaded exosomes were co-cultured with RAW macrophages for 12 h. Subsequently, cells were washed to remove the free-floating exosomes and cells were cultured in the fresh DMEM high glucose medium for 24 h. As a positive control, some cells were treated with LPS (10 ng/ml) for 24 h. At the end of stimulation, cells were washed with 1XPBS twice and cells were lysed in Qiazole (Qiagen, USA) and processed for total RNA extraction (Zymo Research, USA).

As shown in FIG. 7, the miR-132 mimic was successfully delivered to the cells (FIG. 7A). After delivery of the mimic to the cells via exosomes, gene expression consistent with an overexpression of miR-132 was seen in the cells including a reduction in SIRT expression (FIG. 7B), and an induction in IL-1β (mRNA and protein) (FIGS. 7C and 7D), MCP1 (mRNA and protein) (FIGS. 7E and 7F), and TGFβ mRNA (FIG. 7G).

### Example 9 - Production of Exosomes for Delivery of miR-132 Inhibitor, LNA-Anti-miR-132

To make exosomes biologically safe and eliminate the unwanted side effects associated with endogenous exosomal cargo, exosomes are engineered to contain minimal endogenous nucleic acids in order to minimize unwanted biological effects associated with endogenous exosomal RNA as previously described in co-owned application no. PCT/US2020/047894.

Exosomes can then be loaded with desired cargos or payloads including but not limited to nucleic acids (e.g., miRNA inhibitor or miRNA or miRNA mimic).

To further optimize the system, exosomes are further engineered to express tissue specific targeting moieties for organ- and cell-specific delivery. Exosomes expressing the αvβ5 integrin were found to specifically bind to Kupffer cells, thus identifying the liver as the target organ for pre-metastatic niche formation (Hoshino et al. 2015). Using these specific tissue moieties for natural exosomes, exosomes are engineered to express chimeric proteins by recombinant means by fusing structural genes (e.g., integrins, antibodies, peptides) of the proteins in suitable expression vectors expressed with the promotor of exosomal CD63 (a ubiquitous marker for exosomes).

The engineered exosomes are then loaded with an LNA-anti-miR-132 and co-cultured with hepatocytes and Kupffer cells. Levels of miR-132 are determined by quantitative real-time polymerase chain reaction and the results show that the microRNA inhibitor is efficiently delivered to cells by engineered exosomes. There is a decrease in microRNA-132 in the recipient cells.

For in vivo delivery, exosomes devoid of endogenous nucleic acids and expressing αvβ5 integrin are loaded with an LNA-anti-miR-132 and injected into mice (IV) described in Examples 1-3, which are a model for CCl₄-induced liver fibrosis.

mi-RNA-132 is quantified in the liver tissue post-treatment at 5 days, 7 days, and 21 days. CCl₄-induced increase in miR-132 is prevented in mice treated with the loaded exosomes. Histological evaluation shows that administration of exosomes loaded with LNA-anti-miR-132 attenuates the fibrosis phenotype of liver tissue in CCl₄ liver injury, as characterized by less fibrous tissue compared to the controls.

A separate experiment is conducted to identify the efficacy of the exosomes loaded with LNA-anti-miR-132 in survival outcomes. A higher survival rate in the exosome -treated group of mice is observed.

1. A method for treating or preventing liver fibrosis, liver cancer and/or liver disease, comprising administering to a subject in need thereof a therapeutically effective amount of the engineered exosome or extracellular vesicle comprising at least one cargo or payload, wherein the cargo or payload is an agent which inhibits microRNA, and wherein the exosome or extracellular vesicle is engineered to be substantially devoid of endogenous nucleic acids by downregulating or inhibiting at least one protein which is involved in sorting or loading nucleic acids into exosomes or extracellular vesicles, and the microRNA to be inhibited is selected from the group consisting of miR-21, miR-29, miR-33, miR-34a, miR-103/107, miR-122, miR-132, miR-181a, miR-192, miR- 221/222, miR-375, miR-802, and combinations thereof.

2. The method of claim 1, wherein the agent which inhibits microRNA is selected from the group consisting of small molecules, an antisense nucleic acid, a locked nucleic acid (LNA), RNA interference (RNAi), small interfering RNA (siRNA), a microRNA inhibitor, other types of modified nucleic acids, and combinations thereof.

3. The method of claim 2, wherein the agent which inhibit microRNA is a combination of siRNA and another agent with inhibits microRNA.

4. The method of claim 2, wherein the LNA is LNA-anti-miR-132.

5. The method of claim 1, wherein the engineered exosome or extracellular vesicle further comprises a moiety which targets liver.

6. The method of claim 5, wherein the moiety targeting liver is αvP5 integrin or asialoglycoprotein.

7. The method of claim 1, wherein the microRNA is miR-132.

8. A method for treating or preventing liver fibrosis, liver cancer, and/or liver disease, comprising administering to a subject in need thereof a therapeutically effective amount of the engineered exosome or extracellular vesicle comprising at least one cargo or payload, wherein the cargo or payload is an agent which activates microRNA, and wherein the exosome or extracellular vesicle is engineered to be substantially devoid of endogenous nucleic acids by downregulating or inhibiting at least one protein which is involved in sorting or loading nucleic acids into exosomes or extracellular vesicles, and the microRNA is selected from the group consisting of miR-146b, miR-148a, miR-181d, miR-197, and combinations thereof.

9. The method of claim 8, wherein the agent which activates microRNA is selected from the group consisting of small molecules, a microRNA, a microRNA mimic (modified or unmodified), a locked nucleic acid (LNA), RNA interference (RNAi), a dCAS/CRISPR activation system for miRNAs, an agent which increases the stability of miRNAs, and combinations thereof.

10. The method of claim 9, wherein the agent which activates the microRNA is a combination of siRNA and a microRNA or a microRNA mimic.

11. The method of claim 8, wherein the engineered exosome or extracellular vesicle further comprises a moiety which targets liver.

12. The method of claim 11, wherein the moiety targeting liver is αvβ5 integrin or asialoglycoprotein.

13. A method for treating or preventing liver fibrosis, liver cancer, and/or liver disease, comprising administering to a subject in need thereof a therapeutically effective amount of an agent which inhibits microRNA, wherein the microRNA to be inhibited is selected from the group consisting of miR-21, miR-29, miR-33, miR-34a, miR-103/107, miR-122, miR-132, miR-181a, miR-192, miR-221/222, miR-375, miR-802, and combinations thereof.

14. The method of claim 13, wherein the agent which inhibits microRNA is selected from the group consisting of small molecules, an antisense nucleic acid, a locked nucleic acid (LNA), RNA interference (RNAi), small interfering RNA (siRNA), a microRNA inhibitor, other types of modified nucleic acids, and combinations thereof.

15. The method of claim 14, wherein the LNA is LNA-anti-miR-132.

16. The method of claim 13, wherein the microRNA is miR-132.

17. A method for treating or preventing liver fibrosis, liver cancer, and/or liver disease, comprising administering to a subject in need thereof a therapeutically effective amount of an agent which activates microRNA, wherein the microRNA is selected from the group consisting of miR-146b, miR -148a, miR-181d, miR-197, and combinations thereof.

18. The method of claim 17, wherein the agent which activates microRNA is selected from the group consisting of small molecules, a microRNA, a microRNA mimic (modified or unmodified), a locked nucleic acid (LNA), RNA interference (RNAi), a dCAS/CRISPR activation system for miRNAs, an agent which increases the stability of miRNAs, and combinations thereof.

19. The method of claim 1, wherein the liver disease is selected from the group consisting of nonalcoholic steatosis hepatitis, Alcoholic Liver Disease (ALC), hepatocellular carcinoma (HCC), non-alcoholic steatohepatitis (NASH), hepatitis C viral infection (HCV), non-alcoholic fatty liver disease (NAFLD), fatty liver disease, cirrhosis, and combinations thereof.

20. An engineered exosome or extracellular vesicle comprising at least one cargo or payload and at least one surface moiety targeting liver tissue, wherein the cargo or payload is selected from the group consisting of a microRNA, a microRNA mimic and a microRNA inhibitor, wherein the microRNA is selected from the group consisting of miR-146b, miR-148a, miR-181d, and miR-197, wherein the microRNA mimic is selected from the group consisting of miR-146b mimic, miR-148a mimic, miR-181d mimic, and miR-197 mimic, wherein the microRNA inhibitor targets a microRNA selected from the group consisting of miR-21, miR-29, miR-33, miR-34a, miR-103/107, miR-122, miR-132, miR-181a, miR-192, miR-221/222, miR-375, and miR-802, and wherein the exosome or extracellular vesicle is engineered to be substantially devoid of endogenous nucleic acids by downregulating or inhibiting at least one protein which is involved in sorting or loading nucleic acids into exosomes or extracellular vesicles.

21. The engineered exosome or extracellular vesicle of claim 20, wherein the surface targeting moiety comprises an integrin, a laminin, an antibody or an antibody fragment, a receptor, a peptide, a component of extracellular matrix, or combinations thereof.

22. The engineered exosome or extracellular vesicle of claim 21, wherein the integrin comprises an αvβ5 integrin.

23. The engineered exosome or extracellular vesicle of claim 20, wherein the microRNA inhibitor is selected from the group consisting of a small molecule, an antisense nucleic acid, and a locked nucleic acid (LN A).

24. The engineered exosome or extracellular vesicle of claim 23, wherein the LNA is LNA-anti-miR-132.

25. The engineered exosome or extracellular vesicle of claim 20, wherein the cargo or pay load is an LNA-anti-miR-132 and a surface targeting moiety is αvβ5 integrin or asialoglycoprotein.
